(19) United States
(12) Patent Application Publication
(10) Pub. No.: US 2024/0226334 A1
(43) Pub. Date: Jul. 11, 2024

(54) CORRECTION OF DUCHENNE MUSCULAR DYSTROPHY MUTATIONS WITH ALL-IN-ONE ADENO-ASSOCIATED VIRUS-DELIVERED SINGLE-CUT CRISPR

(71) Applicant: The Board of Regents of The University of Texas System, Austin, TX (US)

(72) Inventors: Eric N. OLSON, Dallas, TX (US); Yu ZHANG, Dallas, TX (US); Rhonda BASSEL-DUBY, Dallas, TX (US)

(73) Assignee: The Board of Regents of The University of Texas System, Austin, TX (US)

(21) Appl. No.: 18/563,790

(22) PCT Filed: May 24, 2022
(86) PCT No.: PCT/US2022/030680

§ 371 Date: Nov. 22, 2023

Related U.S. Application Data
(60) Provisional application No. 63/192,801, filed on May 25, 2021.

(51) Int. Cl. A61K 48/00; A61P 21/00; C12N 9/22; C12N 15/11; C12N 15/86

(52) U.S. Cl. CPC A61K 48/0058; A61P 21/00; C12N 9/22; C12N 15/111; C12N 15/86; C12N 2310/20; C12N 2310/315; C12N 2310/321; C12N 2310/322; C12N 2750/14143

(57) ABSTRACT

Provided herein are gene therapy methods, vectors and constructs for the treatment of Duchenne Muscular Dystrophy in a subject.

### PRIORITY CLAIM

This application claims benefit of priority to U.S. Provisional Application Serial No. 63/192,801, filed May 25, 2021, the entire contents of which are hereby incorporated by reference.

### ACKNOWLEDGEMENT OF GOVERNMENT SUPPORT

This invention was made with government support under Grant No. HD087351 awarded by the National Institutes of Health. The government has certain rights in this invention.

### BACKGROUND

### 1. Field

The present disclosure is generally directed to gene therapy vectors and constructs for the treatment of Duchenne Muscular Dystrophy in a subject.

2. Discussion of Related Art

Duchenne muscular dystrophy (DMD), caused by mutations in the X-linked dystrophin gene, is a lethal neuromuscular disease. The DMD gene encodes the dystrophin protein, which is a large cytoskeletal protein essential for tethering the intracellular actin cytoskeleton and extracellular laminin. Absence of dystrophin protein in striated muscles causes skeletal muscle degeneration and myocardial fibrosis, and ultimately progresses to fatal respiratory and cardiac failure. With no transformative treatment available, there is an urgent need to develop new therapeutic approaches for DMD. Correction of DMD mutations in animal models has been attempted by CRISPR/Cas9 genome editing using Streptococcus pyogenes Cas9 (SpCas9) delivered by adeno-associated virus (AAV). However, due to the limited viral packaging capacity of AAV, two AAV vectors are required to deliver the SpCas9 nuclease and its single guide RNA (sgRNA), impeding its therapeutic application. Other studies using a different Cas9 ortholog from Staphylococcus aureus (SaCas9) rely on inducing two DNA double stranded breaks (DSB) in the DMD gene, resulting in additional unwanted genomic modifications, including inversions and AAV integration. Moreover, if one DNA DSB is rejoined by non-homologous end joining (NHEJ) repair before the initiation of the second DNA DSB, the mutant exon cannot be excised, rendering this “double cut” strategy ineffective mammalian innate immune system controls viral infection in part through the actions of protein-based antiviral effectors.

The present disclosure is based on, in part, the surprising discovery of a unique CRISPR-SaCas9 mediated “single cut” gene editing tool to edit DMD mutations in vitro and in vivo. Prior to the present disclosure, SaCas9 had only been used in double cut gene editing applications. Exemplary examples herein describe an efficient single cut gene editing method using a compact Staphylococcus aureus Cas9 (SaCas9) to restore the open reading frame of exon 51, the most commonly affected out-of-frame exon in DMD. Editing of exon 51 in cardiomyocytes derived from human induced pluripotent stem cells revealed a strong preference for exon reframing via a two-nucleotide deletion. Further examples show the adaptation of this system to express SaCas9 and sgRNA from a single AAV9 vector. Systemic delivery of this All-In-One AAV9 system restored dystrophin expression and improved muscle contractility in a mouse model of DMD with exon 50 deletion. Accordingly, the present disclosure herein demonstrates the effectiveness of CRISPR/SaCas9 delivered by a consolidated AAV delivery system in the correction of DMD in vivo, representing a promising therapeutic approach to correct the genetic causes of DMD.

### BRIEF SUMMARY

In accordance with an aspect of the disclosure, provided are gene therapy vectors and constructs comprising nucleic acids encoding a saCas9 endonuclease and an sgRNA targeting a dystrophin gene.

This disclosure provides a gene editing system comprising a nucleic acid encoding a saCas9, an sgRNA or multiple copies of the same sgRNA, and an AAV vector, and methods of using such system. Uses include making a single genome edit in exon 51 of the DMD gene, thereby restoring the open reading frame of exon 51, and treating or ameliorating the symptoms of DMD.

In some embodiments, the system and method further comprise a KKH variant of SaCas9 or a nucleic acid encoding the same.

In some embodiments, the KKH variant of SaCas9 comprises the amino acid sequence of SEQ ID NO: 41.

In some embodiments, the system and method further comprise a HF variant of SaCas9 or a nucleic acid encoding the same.

In some embodiments, the HF variant of SaCas9 comprises the amino acid sequence of SEQ ID NO: 42.

In some embodiments, the system and method further comprise a KKH-HF variant of SaCas9 or a nucleic acid encoding the same.

In some embodiments, the KKH-HF variant of SaCas9 comprises the amino acid sequence of SEQ ID NO: 43.

In some embodiments, the system and method further comprise SaCas9 or a nucleic acid encoding the same.

In some embodiments, the SaCas9 comprises the amino acid sequence of SEQ ID NO: 40.

In some embodiments, the sgRNA is modified.

In some embodiments, the modification alters one or more 2′ positions and/or phosphodiester linkages.

In some embodiments, the modification alters one or more, or all, of the first three nucleotides of the guide RNA.

In some embodiments, the modification alters one or more, or all, of the last three nucleotides of the guide RNA.

In some embodiments, the modification includes one or more of a phosphorothioate modification, a 2′-OME modification, a 2′-O-MOE modification, a 2′-F modification, a 2′-O-methine-4′ bridge modification, a 3′-thiophosphonoacetate modification, or a 2′-deoxy modification.

In some embodiments, the system and method further comprise a pharmaceutically acceptable excipient.

In some embodiments, the system and method are associated with a viral vector.

In some embodiments, the system and method is associated with a viral vector, wherein the viral vector is an adeno-associated virus (AAV) vector, and wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

In some embodiments, the system and method is associated with a viral vector, wherein the viral vector is an adeno-associated virus (AAV) vector, and wherein the AAV vector is an AAV serotype 9 (AAV9) vector.

In some embodiments, the system and method is associated with a viral vector, wherein the viral vector is an adeno-associated virus (AAV) vector, and wherein the AAV vector is an AAVrh10 vector.

In some embodiments, the system and method is associated with a viral vector, wherein the viral vector is an adeno-associated virus (AAV) vector, and wherein the AAV vector is an AAVrh74 vector.

In some embodiments, the system and method is associated with a viral vector, wherein the viral vector comprises a tissue-specific promoter.

In some embodiments, the system and method is associated with a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.

In some embodiments, the system and method is associated with a viral vector, wherein the viral vector comprises any one or more of the following promoters: U6, H1, and 7SK promoter.

In some embodiments, the system and method further comprise a scaffold sequence.

In some embodiments, the scaffold sequence for the sgRNA comprises the sequence of SEQ ID NO: 39.

This disclosure provides a composition comprising a single-molecule guide RNA (sgRNA) comprising a spacer sequence, or a nucleic acid encoding the sgRNA, wherein:
a. the spacer sequence comprises the reverse complement of the “sgRNA DMD Ex51” shown in FIG. 1B; or
b. the spacer sequence recognizes a 5′-AACAGT-3′ PAM in exon 51 as shown in FIG. 1B; or
c. the spacer sequence comprises ACTCTGGTGACACAACCTGTG (SEQ ID NO: 37); or
d. the sgRNA targets TGAGACCACTGTGTTGGACAC (SEQ ID NO: 39); or
e. the sgRNA generates a DNA double-stand break 4-bp upstream of the premature termination codon as shown in FIG. 1B.

In some embodiments, the composition further comprises a KKH variant of SaCas9 or a nucleic acid encoding the same.

In some embodiments, the KKH variant of SaCas9 comprises the amino acid sequence of SEQ ID NO: 41.

In some embodiments, the composition further comprises a HF variant of SaCas9 or a nucleic acid encoding the same.

In some embodiments, the HF variant of SaCas9 comprises the amino acid sequence of SEQ ID NO: 42.

In some embodiments, the composition further comprises a KKH-HF variant of SaCas9 or a nucleic acid encoding the same.

In some embodiments, the KKH-HF variant of SaCas9 comprises the amino acid sequence of SEQ ID NO: 43.

In some embodiments, the composition further comprises SaCas9 or a nucleic acid encoding the same.

In some embodiments, the SaCas9 comprises the amino acid sequence of SEQ ID NO: 40.

In some embodiments, the sgRNA is modified.

In some embodiments, the modification alters one or more 2′ positions and/or phosphodiester linkages.

In some embodiments, the modification alters one or more, or all, of the first three nucleotides of the guide RNA.

In some embodiments, the modification alters one or more, or all, of the last three nucleotides of the guide RNA.

In some embodiments, the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.

In some embodiments, the composition further comprises a pharmaceutically acceptable excipient.

In some embodiments, the composition is associated with a viral vector.

In some embodiments, the composition is associated with a viral vector, wherein the viral vector is an adeno-associated virus (AAV) vector, and wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

In some embodiments, the composition is associated with a viral vector, wherein the viral vector is an adeno-associated virus (AAV) vector, and wherein the AAV vector is an AAV serotype 9 (AAV9) vector.

In some embodiments, the composition is associated with a viral vector, wherein the viral vector is an adeno-associated virus (AAV) vector, and wherein the AAV vector is an AAVrh10 vector.

In some embodiments, the composition is associated with a viral vector, wherein the viral vector is an adeno-associated virus (AAV) vector, and wherein the AAV vector is an AAVrh74 vector.

In some embodiments, the composition is associated with a viral vector, wherein the viral vector comprises a tissue-specific promoter.

In some embodiments, the composition is associated with a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.

In some embodiments, the composition is associated with a viral vector, wherein the viral vector comprises any one or more of the following promoters: U6, H1, and 7SK promoter.

In some embodiments, the composition further comprises a scaffold sequence.

In some embodiments, the scaffold sequence for the sgRNA comprises the sequence of SEQ ID NO: 39.

A method of treating Duchenne Muscular Dystrophy (DMD), the method comprising delivering to a cell the composition of any one of the preceding claims.

This disclosure provides a method of treating Duchenne Muscular Dystrophy (DMD), the method comprising delivering to a cell a composition comprising a single-molecule guide RNA (sgRNA) comprising a spacer sequence, or a nucleic acid encoding the sgRNA, wherein:
a. the spacer sequence comprises the reverse complement of the “sgRNA DMD Ex51” shown in FIG. 1B; or
b. the spacer sequence recognizes a 5′-AACAGT-3′ PAM in exon 51 as shown in FIG. 1B; or
c. the spacer sequence comprises ACTCTGGTGACACAACCTGTG (SEQ ID NO: 37); or
d. the sgRNA targets TGAGACCACTGTGTTGGACAC (SEQ ID NO: 38); or
e. the sgRNA generates a DNA double-stand break 4-bp upstream of the premature termination codon as shown in FIG. 1B.

In some embodiments, the composition is delivered to the cell on a single vector.

In some embodiments, the method further comprises a KKH variant of SaCas9 or a nucleic acid encoding the same.

In some embodiments, the KKH variant of SaCas9 comprises the amino acid sequence of SEQ ID NO: 41.

In some embodiments, the method further comprises a HF variant of SaCas9 or a nucleic acid encoding the same.

In some embodiments, the HF variant of SaCas9 comprises the amino acid sequence of SEQ ID NO: 42.

In some embodiments, the method further comprises a KKH-HF variant of SaCas9 or a nucleic acid encoding the same.

In some embodiments, the KKH-HF variant of SaCas9 comprises the amino acid sequence of SEQ ID NO: 43.

In some embodiments, the method further comprises a SaCas9 or a nucleic acid encoding the same.

In some embodiments, the SaCas9 comprises the amino acid sequence of SEQ ID NO: 40.

In various aspects, the gene therapy vectors and constructs comprise adeno-associated virus (AAV). In various aspects, constructs encoding for the saCas9 endonuclease and the sgRNA targeting the dystrophin gene are packaged in the same AAV vector.

In various aspects, the saCas9 endonuclease and sgRNA, when expressed in a target cell, induce a single double stranded break (DSB) in the dystrophin gene, resulting in an insertion or deletion that restores an open reading frame in the dystrophin gene, allowing expression of functional dystrophin in the cell. In some embodiments, the double stranded break occurs upstream of a premature stop codon in a mutated dystrophin gene. In various embodiments, the premature stop codon is in exon 51 of a native dystrophin gene. In various embodiments, the premature stop codon is results from a deletion of one or more exons 48 to 50 in a native dystrophin gene.

In various embodiments, the saCas9 endonuclease and sgRNA, when expressed in a target cell, induce 2 nucleotide deletion that reframes (e.g., restores) an open reading frame in exon 51. In other embodiments, the saCas9 endonuclease and sgRNA, when expressed in a target cell induces a deletion comprising a slice acceptor, resulting in the complete deletion of exon 51 from the expressed dystrophin.

Also provided are pharmaceutical compositions comprising any of the vectors and constructs expressing the saCas9 endonuclease and sgRNA described herein.

Additional aspects of the disclosure encompass methods for treating Duschenne muscle dystrophy (DMD) in a subject in need thereof, comprising administering to the subject a pharmaceutical composition comprising vectors and/or constructs that enable the expression of the saCas9 endonuclease and sgRNA in a target cell or tissue. In various aspects, the vectors and/or constructs are administered systemically. In some embodiments, the vectors and/or constructs are administered in an AAV vector. In various embodiments, the target cell or tissue is a muscle or cardiovascular (e.g., heart) cell or tissue. In various embodiments the subject is a human.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

Embodiments of the present inventive concept are illustrated by way of example in which like reference numerals indicate similar elements and in which:

FIGS. 1A-1D. Strategies for CRISPR KKH SaCas9-mediated gene editing of human DMD exon 51. (FIG. 1A) An out-of-frame deletion of human DMD exons 48 to 50 (ΔEx48-50) results in splicing of exon 47 to 51, generating a premature termination codon in exon 51. A “single cut” editing strategy was designed to enable CRISPR-KKH SaCas9 DNA cutting to restore the open reading frame of the DMD gene. Small insertions and deletions (INDELs) with two nucleotide deletions (3n−2) can reframe exon 51. Large INDELs disrupting the 5′-AG-3′ splice acceptor sequence cause exon 51 skipping, resulting in splicing of exon 47 to exon 52. (FIG. 1B) Illustration of the sgRNA targeting human DMD exon 51. This sgRNA recognizes a 5′-AACAGT-3′ PAM in exon 51 and generates a DSB 4 base pairs upstream of the 5′-TGA-3′ premature termination sequence (indicated in red). The 5′-AG-3′ splice acceptor sequence is indicated in yellow. (FIG. 1C) Illustration of a plasmid encoding KKH SaCas9 with 2A-GFP, driven by a hybrid form of cytomegalovirus and chicken beta-actin promoter (CBh). The plasmid also encodes a sgRNA driven by the U6 promoter. Cells transfected with this plasmid express GFP, allowing for selection of KKH SaCas9- expressing cells by FACS. (FIG. 1D) Analysis of genomic INDELs in KKH SaCas ΔEx48-50 iPSCs. Productive editing is defined as INDELs with 3n−2 deletion, which are capable of reframing or skipping exon 51. Data are represented as mean ±SEM. Unpaired two-tailed Student's t tests was performed. *P<0.05 (n=3).

FIGS. 2A-F. Restoration of dystrophin expression in DMD ΔEx48-50 cardiomyocytes after CRISPR-KKH SaCas9-mediated “single cut” gene editing. (FIG. 2A) DMD ΔEx48-50 iPSCs were edited by KKH SaCas9 (corrected DMD iPSCs) and then differentiated into corrected cardiomyocytes (CMs) for downstream analysis. (FIG. 2B) Immunocytochemistry shows dystrophin restoration in mixtures of DMD ΔEx48-50 CMs following KKH SaCas9-mediated “single cut” gene editing. Red, dystrophin staining; green, troponin I staining. Scale bar, 100 μm. (FIG. 2C) Western blot shows dystrophin restoration in mixtures of DMD ΔEx48-50 CMs following KKH SaCas9-mediated “single cut” gene editing. Dilutions of protein extract from healthy control CMs were used to standardize dystrophin protein expression. Vinculin was used as the loading control. (FIG. 2D) Representative traces of spontaneous calcium activity of iPSC-derived CMs cultured with calcium indicator Fluo-4AM. Traces show change in fluorescence intensity (F) in relationship to resting fluorescence intensity (Fo). (FIG. 2E) Quantification of calcium release phase of contraction, as measured by time to peak, in iPSC-derived CMs. Data are represented as mean ±SEM. One-way ANOVA was performed with post-hoc Tukey's multiple comparisons test. ****P<0.0001 (n=40). (FIG. 2F) Quantification of calcium reuptake phase of contraction, as measured by tau, in iPSC-derived CMs. Data are represented as mean ±SEM. One-way ANOVA was performed with post-hoc Tukey's multiple comparisons test. ****P<0.0001 (n=40).

FIGS. 3A-C. Systemic delivery of All-In-One AAV-packaged KKH SaCas9 restores dystrophin expression in ΔEx50 mice. (FIG. 3A) Illustration of the All-In-One AAV vector used to deliver KKH SaCas9 gene editing components. KKH SaCas9 expression is driven by a muscle specific CK8 promoter. Two copies of the same sgRNA targeting mouse Dmd exon 51 are driven by two RNA polymerase III promoters, 7SK and U6. (FIG. 3B) Illustration of systemic delivery of All-In-One AAV vectors in ΔEx50 mice. Postnatal day 4 ΔEx50 mice were injected intraperitoneally with 2×10¹⁴ or 4×10¹⁴ vg/kg of All-In-One AAV vectors. Four weeks after systemic delivery, ΔEx50 mice and WT littermates were dissected for analysis. (FIG. 3C) Immunohistochemistry shows restoration of dystrophin in the tibialis anterior (TA), triceps, diaphragm, and heart of ΔEx50 mice 4 weeks after systemic delivery of AAV-packaged KKH SaCas9 and sgRNA. Dystrophin is shown in green. n=6 for each muscle group. Scale bars, 100 μm.

FIGS. 4A-C. Western blot and genomic analysis of skeletal muscles and heart of ΔEx50 mice receiving systemic All-In-One AAV delivery of KKH SaCas9 gene editing components. (FIG. 4A) Western blot analysis shows restoration of dystrophin expression in the TA, triceps, diaphragm, and heart of ΔEx50 mice 4 weeks after systemic delivery of All-In-One AAV- packaged KKH SaCas9 and sgRNA. The dose of the AAV vector is indicated. Dilutions of protein extract from WT mice were used to standardize dystrophin protein expression. Vinculin was used as the loading control (n=3 for each dose). (FIG. 4B) Quantification of dystrophin expression in the TA, triceps, diaphragm, and heart. Relative dystrophin intensity was calibrated with vinculin internal control before normalizing to the WT control. (FIG. 4C) Genomic INDEL quantification by deep sequencing analysis of the TA, triceps, diaphragm, and heart of ΔEx50 mice 4 weeks after systemic delivery of All-In-One AAV- packaged KKH SaCas9 and sgRNA.

FIGS. 5A-F. Systemic delivery of All-In-One AAV-packaged CRISPR-KKH SaCas9 improves muscle function in ΔEx50 mice. (FIG. 5A and FIG. 5B) Specific force (mN/mm²) of the soleus (FIG. 5A) and extensor digitorum longus (EDL) (FIG. 5B) in WT, ΔEx50 mice untreated, and ΔEx50 mice treated with All-In-One AAV-packaged KKH SaCas9. Data are represented as mean ±SEM. Brown-Forsythe and Welch ANOVA test was performed. *P<0.05, **P<0.005 (n=6). (FIG. 5C and FIG. 5D) Maximal tetanic force of the soleus (FIG. 5C) and EDL (FIG. 5D) in WT, ΔEx50 mice untreated, and ΔEx50 mice treated with All-In-One AAV-packaged KKH SaCas9. (FIG. 5E and FIG. 5F) Fatigue resistance analysis of soleus (FIG. 5E) and EDL (FIG. 5F) in WT, ΔEx50 mice untreated, and ΔEx50 mice treated with All-In-One AAV-packaged KKH SaCas9. Data are represented as mean ±SEM. Nonlinear Regression with Extra sum-of-squares F Test was performed (n=6).

FIG. 6. INDEL analysis of KKH SaCas9-edited DMD ΔEx48-50 iPSCs. Analysis of genomic INDELs in KKH SaCas9-edited DMD ΔEx48-50 iPSCs shows high frequency of 5′-CT-3′ dinucleotide deletion. Microhomology sequence is highlighted in red.

FIGS. 7A-C. RT-PCR analysis of KKH SaCas9-edited DMD ΔEx48-50 iPSCs. (FIGS. 7A-C) RT-PCR analysis of uncorrected DMD iPSC-derived cardiomyocytes (FIG. 7A), KKH SaCas9-edited cardiomyocytes (FIG. 7B) and healthy control cardiomyocytes (FIG. 7C). Uncorrected DMD iPSC-derived cardiomyocytes have a 5′-TGA-3′ premature termination codon in exon 51. After KKH SaCas9-mediated reframing, the ORF of dystrophin is restored.

FIGS. 8A-B. Off-target analysis of KKH SaCas9-edited DMD ΔEx48-50 iPSCs. (FIG. 8A) Genomic deep sequencing analysis on the top 8 predicted off-target sites of KKH SaCas9 sgRNA. (FIG. 8B) Percentage of genomic INDEL in deep sequencing analysis on the top eight predicted off-target sites of KKH SaCas9 sgRNA.

FIG. 9. Whole muscle scanning of immunohistochemistry of TA, triceps, diaphragm, and heart of KKH SaCas9-corrected ΔEx50 mice. Whole muscle scanning of TA, triceps, diaphragm, and heart of ΔEx50 mice 4 weeks after systemic delivery of All-In-One AAV-packaged KKH SaCas9 and sgRNA. The dose of All-In-One AAV vector is shown in the figure. Dystrophin is shown in green.

FIGS. 10A-C. Quantification of percentage of dystrophin positive myofibers. Quantification of percentage of dystrophin positive myofibers in TA, triceps, and diaphragm of KKH SaCas9-corrected ΔEx50 mice. The dose of All-In-One AAV-packaged KKH SaCas9 is shown in the figure. Data are represented as mean ±SEM. One-way ANOVA was performed with post-hoc Tukey's multiple comparisons test. *P<0.05, **P<0.01, ****P<0.0001 (n=6).

FIG. 11. Muscle histology of ΔEx50 mice after systemic delivery of All-In-One AAV-packaged KKH SaCas9 and sgRNA. H&E staining of TA, triceps, diaphragm, and heart of ΔEx50 mice 4 weeks after systemic delivery of All-In-One AAV-packaged KKH SaCas9 and sgRNA. The dose of All-In-One AAV vector is shown in the figure. n =6 for each muscle group. Scale bar, 100 μm.

FIG. 12. Whole muscle scanning of H&E staining of TA, triceps, diaphragm, and heart of KKH SaCas9-corrected ΔEx50 mice. Whole muscle scanning of H&E staining of TA, triceps, diaphragm, and heart of ΔEx50 mice 4 weeks after systemic delivery of All-In-One AAV-packaged KKH SaCas9 and sgRNA. The dose of All-In-One AAV vector is shown in the figure.

FIGS. 13A-D. Quantification of histological improvement of ΔEx50 mice after systemic delivery of All-In-One AAV-packaged KKH SaCas9 and sgRNA. (FIGS. 13A-C) Quantification of percentage of centrally nucleated myofibers of TA (FIG. 13A), triceps (FIG. 13B), and diaphragm (FIG. 13C) of ΔEx50 mice 4 weeks after systemic delivery of All-In-One AAV- packaged KKH SaCas9 and sgRNA. The dose of All-In-One AAV vector is shown in the figure. Data are represented as mean ±SEM. One-way ANOVA was performed with post-hoc Tukey's multiple comparisons test. *P<0.05, **P<0.01, ***P<0.005 (n=6). (FIG. 13D) Quantification of fiber size of TA muscle of ΔEx50 mice 4 weeks after systemic delivery of All-In-One AAV-packaged KKH SaCas9 and sgRNA. The dose of All-In-One AAV vector is shown in the figure. Data are represented as mean ±SEM.

FIG. 14. Masson trichrome staining of ΔEx50 mice after systemic delivery of All-In-One AAV-packaged KKH SaCas9 and sgRNA. Masson trichrome staining of TA, triceps, diaphragm, and heart of ΔEx50 mice 4 weeks after systemic delivery of All-In-One AAV-packaged KKH SaCas9 and sgRNA. The dose of All-In-One AAV vector is shown in the figure. Scale bars, 100 μm.

FIG. 15. Whole muscle scanning of Masson trichrome staining of TA, triceps, diaphragm, and heart of KKH SaCas9-corrected ΔEx50 mice. Whole muscle scanning of Masson trichrome staining of TA, triceps, diaphragm, and heart of ΔEx50 mice 4 weeks after systemic delivery of All-In-One AAV-packaged KKH SaCas9 and sgRNA. The dose of All-In-One AAV vector is shown in the figure.

FIGS. 16A-C. Quantification of muscle fibrotic/necrotic area of ΔEx50 mice after systemic delivery of All-In-One AAV-packaged KKH SaCas9 and sgRNA. (FIGS. 16A-C) Quantification of percentage of fibrosis/necrosis of TA (FIG. 16A), triceps (FIG. 16B), and diaphragm (FIG. 16C) of ΔEx50 mice 4 weeks after systemic delivery of All-In-One AAV-packaged KKH SaCas9 and sgRNA. The dose of All-In-One AAV vector is shown in the figure. Data are represented as mean ±SEM. One-way ANOVA was performed with post-hoc Tukey's multiple comparisons test. * P<0.05, **P<0.01, ***P<0.005 (n=6).

FIGS. 17A-B. Grip strength analysis of ΔEx50 mice after systemic delivery of All-In-One AAV-packaged KKH SaCas9 and sgRNA. (FIG. 17A and FIG. 17B) Grip strength analysis of forelimb (FIG. 17A) and hindlimb (FIG. 17B) of WT, ΔEx50 mice untreated, and ΔEx50 mice 4 weeks after systemic delivery of All-In-One AAV-packaged KKH SaCas9 and sgRNA. The dose of All-In-One AAV vector is shown in the figure. Grams of force is normalized with body weight. Data are represented as mean ±SEM. One-way ANOVA was performed with post-hoc Tukey's multiple comparisons test. *P<0.05, **P<0.01 (n=6).

FIGS. 18A-D. Histological and genomic INDEL analysis of soleus and EDL muscles of ΔEx50 mice after systemic delivery of All-In-One AAV-packaged KKH SaCas9 and sgRNA. (FIG. 18A and FIG. 18B) Immunohistochemistry (FIG. 18A) and H&E staining (FIG. 18B) of soleus and EDL muscles of WT, ΔEx50 mice untreated, and ΔEx50 mice 4 weeks after systemic delivery of All-In-One AAV-packaged KKH SaCas9 and sgRNA. The dose of All-In-One AAV vector is shown in the figure. Scale bars, 100 μm. (FIG. 18C) Genomic INDEL analysis of soleus and (FIG. 18D) EDL muscles of WT, ΔEx50 mice untreated, and ΔEx50 mice 4 weeks after systemic delivery of All-In-One AAV-packaged KKH SaCas9 and sgRNA. The dose of All-In-One AAV vector is shown in the figure. Data are represented as mean ±SEM. Unpaired two-tailed Student's t tests was performed. *P<0.05 (n=3).

FIG. 19. Serum creatine kinase (CK) analysis of KKH SaCas9-edited ΔEx50 mice. Serum CK was measured in WT, ΔEx50 mice untreated, and ΔEx50 mice 4 weeks after treatment with All-In-One AAV-packaged KKH SaCas9 and sgRNA. The dose of All-In-One AAV vector is shown in the figure. Serum CK was normalized to WT mice and shown as fold expression. Data are represented as mean ±SEM. One-way ANOVA was performed with post-hoc Tukey's multiple comparisons test. **P<0.005, ***P<0.001 (n=6).

The drawing figures do not limit the present inventive concept to the specific embodiments disclosed and described herein. The drawings are not necessarily to scale emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part on, the surprising discovery of a unique CRISPR-SaCas9 mediated “single cut” gene editing tool to edit DMD mutations in vitro and in vivo. The methods and compositions herein, in some embodiments, employ a single vector strategy to deliver a compact Staphylococcus aureus Cas9 (SaCas9) and a gRNA to tissue to restore the open reading frame of exon 51, the most commonly affected out-of-frame exon in DMD. Accordingly, the present disclosure herein avoids two common limitations in DMD gene therapeutics—multi-vector protocols and reliance on “double cut” gene editing tools. The result is a simpler, more precise therapeutic approach to correct the genetic causes of DMD in vivo.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all value from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. Compositions

Provided herein is a gene vector or construct comprising a nucleotide sequence encoding for a Cas9 protein and/or a nucleotide sequence encoding for a sgRNA targeting a dystrophin gene.

In various embodiments, the Cas9 protein is derived from a Staphylococcus aureus Cas9. In further embodiments, the Cas9 protein comprises a modified Cas9 protein having a modified protospacer adjacent motif (PAM)-interacting domain. In various embodiments, the modified Cas9 protein can comprise at least 1, at least 2, or at least 3 substitutions in a PAM interacting domain wherein the inclusion of these substitutions increase the genome editing activities at a target site comprising a 5′-NNNRRT-3′ PAM, where “N” is adenine, guanine, cytosine or thymine and “R” is guanine or adenine. For example, the Cas9 protein can comprise a KKH SaCas9.

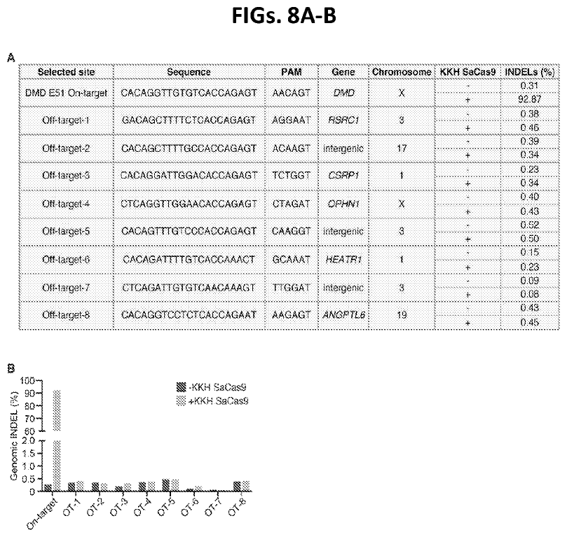

In various embodiments, the sgRNA targeting a dystrophin gene targets an exon having a mutation in subjects suffering from Duschenne muscular dystrophy (DMD). In various embodiments, the mutation comprises a deletion of one or more exons in a native dystrophin gene causing a premature termination codon in a downstream exon. In various embodiments, the deletion comprises a deletion of one or more of exons 48-50 in a native dystrophin gene. In various embodiments, the downstream exon having a premature stop codon as a result of the deletion of one or more of exons 48 to 50 is exon 51. In various embodiments, the sgRNA targets a 5′-AACAGT-3′ PAM in exon 51. In various embodiments, the sgRNA comprises a nucleic acid provided in FIG. 8A. In further embodiments, the sgRNA targets a PAM comprising a nucleic acid sequence provided in FIG. 8A.

In various embodiments, the dystrophin gene is a human gene. Accordingly, the exon and PAMs targeted herein can correspond to the human exon 51 for dystrophin.

In various embodiments, when the gene vector or construct is expressed in a cell, the saCas9 protein and sgRNA facilitate a single double stranded break (DSB) upstream (e.g., about 4 bp upstream) of a premature termination codon in exon 51, which can be repaired endogenously using non-homologous end-joining (NHEJ). In various embodiments, the repair can result in an insertion or deletion (INDEL) which either reframes exon 51 (allowing continued transcription across the mutated stop codon), or deletes a splice acceptor (e.g., a 5′-AG-3′ splice acceptor) resulting in removal of exon 51 and transcription of the rest of the dystrophin gene. In various embodiments, the insertion or deletion comprises a 2 nucleotide deletion that reframes exon 51. In other embodiments, the insertion or deletion comprises a deletion comprising the 5′-AG-3′ splice acceptor, resulting in a deletion in exon 51.

In various embodiments, the gene vector or construct may be delivered to a target tissue via an adeno-associated virus (AAV) vector. Exemplary AAV vectors that can be used to deliver gene vectors or constructs include recombinant adeno-associated virus serotype 2 or recombinant adeno-associated virus serotype 5. Alternatively, other viral vectors, such as herpes simplex virus, can be used for delivery of the nucleic acid to the target cell. In some examples, non-viral vectors, such as but not limited to, plasmid DNA delivered alone or complexed with liposomal compounds or polyethyleneamine, may be used to deliver the gene vector or construct to the target tissue.

In various embodiments, the gene vector or construct can comprise additional controller sequences (e.g., promoters, terminators, restriction sites, etc) that facilitate the expression of the Cas9 protein and the sgRNA only in certain cells. For example, a muscle or heart specific promoter can be included in the gene vector or construct to facilitate expression in muscle or heart tissue. For example, the muscle-specific CK8 promoter can be included.

In various embodiments, an “all-in-one” gene therapy system is provided, wherein nucleic acids encoding Cas9 and sgRNA are packaged in the same AAV vector. In some embodiments, multiple copies of the nucleic acid encoding the sgRNA are provided in the AAV vector. For example, the AAV vector can contain 1, 2, 3, 4 or more cassettes encoding the sgRNA. In some embodiments, the AAV vector contains 2 cassettes encoding the sgRNA and 1 cassette encoding the Cas9 endonuclease.

In various embodiments, any of the gene vectors, constructs, AAV vectors or other components described herein can be prepared as a pharmaceutical composition. As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the nucleic acid molecule that encodes for, or allows for the expression of, the Cas9 endonuclease (e.g., SaCas9) and the sgRNA.

In various embodiments, the nucleic acid sequence encoding for the Cas9 endonuclease and the nucleic acid sequence encoding the sgRNA are included in a single construct and packaged in a single AAV vector.

### Pharmaceutically Acceptable Carriers and Excipients

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

In various embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference herein in its entirety.

In various embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In various embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition.

In various embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In some embodiments, suitable thickening agent may be carboxymethylcellulose.

In various embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the present disclosure. In s aspects, additives will be biocompatible, and will not be harsh, abrasive, or allergenic.

In various embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In various embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

Each of the guide sequences shown in Table 2 may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The guide RNA can be considered to comprise a scaffold sequence necessary for endonuclease binding and a spacer sequence required to bind to the genomic target sequence.

In some embodiments, an exemplary scaffold sequence suitable for use with SaCas9 may be used. In some embodiments, the SaCas9 scaffold to follow the guide sequence at its 3′ end is referred to as “SaScaffoldV2” and is: GTTTAAGTACTCTGTGCTGGAAACAGCACAGAATCTACTTAAACAAGGCAAAATGCCGT GTTTATCTCGTCAACTTGTTGGCGAGAT (SEQ ID NO: 39) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 39, or a sequence that differs from SEQ ID NO: 39 by no more than 1, 2, 3, 4, 5, 10, 15, 20, or 25 nucleotides.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 40:

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLNSEL
TQEEIEQISNLKGYTGTHNLSLKAINLILDELWHTNDNQIAIFNRLKLVPKKVDLSQQKEIPTT
LVDDFILSPVVKRSFIQSIKVINAIIKKYGLPNDIIIELAREKNSKDAQKMINEMQKRNRQTNER
IEEIIRTTGKENAKYLIEKIKLHDMQEGKCLYSLEAIPLEDLLNNPFNYEVDHIIPRSVSFDNSF
NNKVLVKQEENSKKGNRTPFQYLSSSDSKISYETFKKHILNLAKGKGRISKTKKEYLLEERDI
NRFSVQKDFINRNLVDTRYATRGLMNLLRSYFRVNNLDVKVKSINGGFTSFLRRKWKFKKE
RNKGYKHHAEDALIIANADFIFKEWKKLDKAKKVMENQMFEEKQAESMPEIETEQEYKEIFI
TPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKK
IKYYGNKLNAHLDITDDYPNSRNKVVKLSLKPYRFDVYLDNGVYKFVTVKNLDVIKKENYYE
VNSKCYEEAKKLKKISNQAEFIASFYNNDLIKINGELYRVIGVNNDLLNRIEVNMIDITYREYL
ENMNDKRPPRIIKTIASKTQSIKKYSTDILGNLYEVKSKKHPQIIKKG.

In some embodiments, the nucleic acid encoding SaCas9 comprises the nucleic acid of SEQ ID NO: 44:

AAGCGCAATTACATCCTGGGCCTGGATATCGGCATCACCTCCGTGGGCTACGG
CATCATCGACTATGAGACACGGGATGTGATCGACGCCGGCGTGAGACTGTTCAAGGA
GGCCAACGTGGAGAACAATGAGGGCCGGCGGAGCAAGAGGGGAGCAAGGCGCCTGA
AGCGGAGAAGGCGCCACAGAATCCAGAGAGTGAAGAAGCTGCTGTTCGATTACAACC
TGCTGACCGACCACTCCGAGCTGTCTGGCATCAATCCTTATGAGGCCCGGGTGAAGG
GCCTGTCCCAGAAGCTGTCTGAGGAGGAGTTTTCTGCCGCCCTGCTGCACCTGGCAA
AGAGGAGAGGCGTGCACAACGTGAATGAGGTGGAGGAGGACACCGGCAACGAGCTG
AGCACAAAGGAGCAGATCAGCCGCAATTCCAAGGCCCTGGAGGAGAAGTATGTGGCC
GAGCTGCAGCTGGAGCGGCTGAAGAAGGATGGCGAGGTGAGGGGCTCCATCAATCG
CTTCAAGACCTCTGACTACGTGAAGGAGGCCAAGCAGCTGCTGAAGGTGCAGAAGGC
CTACCACCAGCTGGATCAGAGCTTTATCGATACATATATCGACCTGCTGGAGACCAGG
CGCACATACTATGAGGGACCAGGAGAGGGCTCCCCCTTCGGCTGGAAGGACATCAAG
GAGTGGTACGAGATGCTGATGGGCCACTGCACCTATTTTCCAGAGGAGCTGAGATCC
GTGAAGTACGCCTATAACGCCGATCTGTACAACGCCCTGAATGACCTGAACAACCTGG
TCATCACCAGGGATGAGAACGAGAAGCTGGAGTACTATGAGAAGTTCCAGATCATCGA
GAACGTGTTCAAGCAGAAGAAGAAGCCTACACTGAAGCAGATCGCCAAGGAGATCCTG
GTGAACGAGGAGGACATCAAGGGCTACCGCGTGACCAGCACAGGCAAGCCAGAGTTC
ACCAATCTGAAGGTGTATCACGATATCAAGGACATCACAGCCCGGAAGGAGATCATCG
AGAACGCCGAGCTGCTGGATCAGATCGCCAAGATCCTGACCATCTATCAGAGCTCCGA
GGACATCCAGGAGGAGCTGACCAACCTGAATAGCGAGCTGACACAGGAGGAGATCGA
GCAGATCAGCAATCTGAAGGGCTACACCGGCACACACAACCTGTCCCTGAAGGCCAT
CAATCTGATCCTGGATGAGCTGTGGCACACAAACGACAATCAGATCGCCATCTTTAACA
GGCTGAAGCTGGTGCCAAAGAAGGTGGACCTGAGCCAGCAGAAGGAGATCCCAACCA
CACTGGTGGACGATTTCATCCTGTCCCCCGTGGTGAAGCGGAGCTTCATCCAGAGCAT
CAAAGTGATCAACGCCATCATCAAGAAGTACGGCCTGCCCAATGATATCATCATCGAG
CTGGCCAGGGAGAAGAACTCTAAGGACGCCCAGAAGATGATCAATGAGATGCAGAAG
AGGAACCGCCAGACCAATGAGCGGATCGAGGAGATCATCAGAACCACAGGCAAGGAG
AACGCCAAGTACCTGATCGAGAAGATCAAGCTGCACGATATGCAGGAGGGCAAGTGT
CTGTATAGCCTGGAGGCCATCCCTCTGGAGGACCTGCTGAACAATCCATTCAACTACG
AGGTGGATCACATCATCCCCCGGAGCGTGAGCTTCGACAATTCCTTTAACAATAAGGT
GCTGGTGAAGCAGGAGGAGAACTCTAAGAAGGGCAATAGGACCCCTTTCCAGTACCT
GTCTAGCTCCGATTCTAAGATCAGCTACGAGACCTTCAAGAAGCACATCCTGAATCTG
GCCAAGGGCAAGGGCCGCATCTCTAAGACCAAGAAGGAGTACCTGCTGGAGGAGCG
GGACATCAACAGATTCAGCGTGCAGAAGGACTTCATCAACCGGAATCTGGTGGACACC
AGATACGCCACACGCGGCCTGATGAATCTGCTGCGGTCCTATTTCAGAGTGAACAATC
TGGATGTGAAGGTGAAGAGCATCAACGGCGGCTTCACCTCCTTTCTGCGGAGAAAGTG
GAAGTTTAAGAAGGAGAGAAACAAGGGCTATAAGCACCACGCCGAGGATGCCCTGAT
CATCGCCAATGCCGACTTCATCTTTAAGGAGTGGAAGAAGCTGGACAAGGCCAAGAAA
GTGATGGAGAACCAGATGTTCGAGGAGAAGCAGGCCGAGAGCATGCCCGAGATCGAG
ACCGAGCAGGAGTACAAGGAGATTTTCATCACACCTCACCAGATCAAGCACATCAAGG
ACTTCAAGGACTACAAGTATTCCCACAGGGTGGATAAGAAGCCCAACCGCGAGCTGAT
CAATGACACCCTGTATTCTACAAGGAAGGACGATAAGGGCAATACCCTGATCGTGAAC
AATCTGAACGGCCTGTACGACAAGGATAATGACAAGCTGAAGAAGCTGATCAACAAGA
GCCCCGAGAAGCTGCTGATGTACCACCACGATCCTCAGACATATCAGAAGCTGAAGCT
GATCATGGAGCAGTACGGCGACGAGAAGAACCCACTGTATAAGTACTATGAGGAGACC
GGCAACTACCTGACAAAGTATTCCAAGAAGGATAATGGCCCCGTGATCAAGAAGATCA
AGTACTATGGCAACAAGCTGAATGCCCACCTGGACATCACCGACGATTACCCCAACAG
CCGGAATAAGGTGGTGAAGCTGAGCCTGAAGCCATACAGGTTCGACGTGTACCTGGA
CAACGGCGTGTATAAGTTTGTGACAGTGAAGAATCTGGATGTGATCAAGAAGGAGAAC
TACTATGAAGTGAATAGCAAGTGCTACGAGGAGGCCAAGAAGCTGAAGAAGATCAGCA
ACCAGGCCGAGTTCATCGCCTCTTTTTACAACAATGACCTGATCAAGATCAATGGCGA
GCTGTATAGAGTGATCGGCGTGAACAATGATCTGCTGAACCGCATCGAAGTGAATATG
ATCGACATCACCTACCGGGAGTATCTGGAGAACATGAATGATAAGAGGCCCCCTCGCA
TCATCAAGACCATCGCCTCTAAGACACAGAGCATCAAGAAGTACTCTACAGACATCCTG
GGCAACCTGTATGAGGTGAAGAGCAAGAAGCACCCTCAGATCATCAAGAAGGGC.

In some embodiments, the SaCas9 is a variant of the amino acid sequence of SEQ ID NO: 40. In some embodiments, the SaCas9 comprises an amino acid other than an E at the position corresponding to position 781 of SEQ ID NO: 40. In some embodiments, the SaCas9 comprises an amino acid other than an N at the position corresponding to position 967 of SEQ ID NO: 40. In some embodiments, the SaCas9 comprises an amino acid other than an R at the position corresponding to position 1014 of SEQ ID NO: 40. In some embodiments, the SaCas9 comprises a K at the position corresponding to position 781 of SEQ ID NO: 40. In some embodiments, the SaCas9 comprises a K at the position corresponding to position 967 of SEQ ID NO: 40. In some embodiments, the SaCas9 comprises an H at the position corresponding to position 1014 of SEQ ID NO: 40. In some embodiments, the SaCas9 comprises an amino acid other than an E at the position corresponding to position 781 of SEQ ID NO: 40; an amino acid other than an N at the position corresponding to position 967 of SEQ ID NO: 40; and an amino acid other than an R at the position corresponding to position 1014 of SEQ ID NO: 40. In some embodiments, the SaCas9 comprises a K at the position corresponding to position 781 of SEQ ID NO: 40; a K at the position corresponding to position 967 of SEQ ID NO: 40; and an H at the position corresponding to position 1014 of SEQ ID NO: 40.

In some embodiments, the SaCas9 comprises an amino acid other than an R at the position corresponding to position 244 of SEQ ID NO: 40. In some embodiments, the SaCas9 comprises an amino acid other than an N at the position corresponding to position 412 of SEQ ID NO: 40. In some embodiments, the SaCas9 comprises an amino acid other than an N at the position corresponding to position 418 of SEQ ID NO: 40. In some embodiments, the SaCas9 comprises an amino acid other than an R at the position corresponding to position 653 of SEQ ID NO: 40. In some embodiments, the SaCas9 comprises an amino acid other than an R at the position corresponding to position 244 of SEQ ID NO: 40; an amino acid other than an N at the position corresponding to position 412 of SEQ ID NO: 40; an amino acid other than an N at the position corresponding to position 418 of SEQ ID NO: 40; and an amino acid other than an R at the position corresponding to position 653 of SEQ ID NO: 40. In some embodiments, the SaCas9 comprises an A at the position corresponding to position 244 of SEQ ID NO: 40. In some embodiments, the SaCas9 comprises an A at the position corresponding to position 412 of SEQ ID NO: 40. In some embodiments, the SaCas9 comprises an A at the position corresponding to position 418 of SEQ ID NO: 40. In some embodiments, the SaCas9 comprises an A at the position corresponding to position 653 of SEQ ID NO: 40. In some embodiments, the SaCas9 comprises an A at the position corresponding to position 244 of SEQ ID NO: 40; an A at the position corresponding to position 412 of SEQ ID NO: 40; an A at the position corresponding to position 418 of SEQ ID NO: 40; and an A at the position corresponding to position 653 of SEQ ID NO: 40.

In some embodiments, the SaCas9 comprises an amino acid other than an R at the position corresponding to position 244 of SEQ ID NO: 40; an amino acid other than an N at the position corresponding to position 412 of SEQ ID NO: 40; an amino acid other than an N at the position corresponding to position 418 of SEQ ID NO: 40; an amino acid other than an R at the position corresponding to position 653 of SEQ ID NO: 40; an amino acid other than an E at the position corresponding to position 781 of SEQ ID NO: 40; an amino acid other than an N at the position corresponding to position 967 of SEQ ID NO: 40; and an amino acid other than an R at the position corresponding to position 1014 of SEQ ID NO: 40. In some embodiments, the SaCas9 comprises an A at the position corresponding to position 244 of SEQ ID NO: 40; an A at the position corresponding to position 412 of SEQ ID NO: 40; an A at the position corresponding to position 418 of SEQ ID NO: 40; an A at the position corresponding to position 653 of SEQ ID NO: 40; a K at the position corresponding to position 781 of SEQ ID NO: 40; a K at the position corresponding to position 967 of SEQ ID NO: 40; and an H at the position corresponding to position 1014 of SEQ ID NO: 40.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 41 (designated herein as SaCas9-KKH or SACAS9KKH):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLNSEL
TQEEIEQISNLKGYTGTHNLSLKAINLILDELWHTNDNQIAIFNRLKLVPKKVDLSQQKEIPTT
LVDDFILSPVVKRSFIQSIKVINAIIKKYGLPNDIIIELAREKNSKDAQKMINEMQKRNRQTNER
IEEIIRTTGKENAKYLIEKIKLHDMQEGKCLYSLEAIPLEDLLNNPFNYEVDHIIPRSVSFDNSF
NNKVLVKQEENSKKGNRTPFQYLSSSDSKISYETFKKHILNLAKGKGRISKTKKEYLLEERDI
NRFSVQKDFINRNLVDTRYATRGLMNLLRSYFRVNNLDVKVKSINGGFTSFLRRKWKFKKE
RNKGYKHHAEDALIIANADFIFKEWKKLDKAKKVMENQMFEEKQAESMPEIETEQEYKEIFI
TPHQIKHIKDFKDYKYSHRVDKKPNRKLINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKK
IKYYGNKLNAHLDITDDYPNSRNKVVKLSLKPYRFDVYLDNGVYKFVTVKNLDVIKKENYYE
VNSKCYEEAKKLKKISNQAEFIASFYKNDLIKINGELYRVIGVNNDLLNRIEVNMIDITYREYL
ENMNDKRPPHIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 42 (designated herein as SaCas9-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELAS
VKYAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLNSEL
TQEEIEQISNLKGYTGTHNLSLKAINLILDELWHTNDAQIAIFARLKLVPKKVDLSQQKEIPTT
LVDDFILSPVVKRSFIQSIKVINAIIKKYGLPNDIIIELAREKNSKDAQKMINEMQKRNRQTNER
IEEIIRTTGKENAKYLIEKIKLHDMQEGKCLYSLEAIPLEDLLNNPFNYEVDHIIPRSVSFDNSF
NNKVLVKQEENSKKGNRTPFQYLSSSDSKISYETFKKHILNLAKGKGRISKTKKEYLLEERDI
NRFSVQKDFINRNLVDTRYATAGLMNLLRSYFRVNNLDVKVKSINGGFTSFLRRKWKFKKE
RNKGYKHHAEDALIIANADFIFKEWKKLDKAKKVMENQMFEEKQAESMPEIETEQEYKEIFI
TPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKK
IKYYGNKLNAHLDITDDYPNSRNKVVKLSLKPYRFDVYLDNGVYKFVTVKNLDVIKKENYYE
VNSKCYEEAKKLKKISNQAEFIASFYNNDLIKINGELYRVIGVNNDLLNRIEVNMIDITYREYL
ENMNDKRPPRIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 43 (designated herein as SaCas9-KKH-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELAS
VKYAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLNSEL
TQEEIEQISNLKGYTGTHNLSLKAINLILDELWHTNDAQIAIFARLKLVPKKVDLSQQKEIPTT
LVDDFILSPVVKRSFIQSIKVINAIIKKYGLPNDIIIELAREKNSKDAQKMINEMQKRNRQTNER
IEEIIRTTGKENAKYLIEKIKLHDMQEGKCLYSLEAIPLEDLLNNPFNYEVDHIIPRSVSFDNSF
NNKVLVKQEENSKKGNRTPFQYLSSSDSKISYETFKKHILNLAKGKGRISKTKKEYLLEERDI
NRFSVQKDFINRNLVDTRYATAGLMNLLRSYFRVNNLDVKVKSINGGFTSFLRRKWKFKKE
RNKGYKHHAEDALIIANADFIFKEWKKLDKAKKVMENQMFEEKQAESMPEIETEQEYKEIFI
TPHQIKHIKDFKDYKYSHRVDKKPNRKLINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKK
IKYYGNKLNAHLDITDDYPNSRNKVVKLSLKPYRFDVYLDNGVYKFVTVKNLDVIKKENYYE
VNSKCYEEAKKLKKISNQAEFIASFYKNDLIKINGELYRVIGVNNDLLNRIEVNMIDITYREYL
ENMNDKRPPHIIKTIASKTQSIKKYSTDILGNLYEVKSKKHPQIIKKG.

### Dosage Formulations

Suitable routes of administration may, for example, include oral, rectal, transmucosal, especially transnasal, intestinal or parenteral delivery, including intramuscular, subcutaneous and intramedullary injections as well as, intravenous, intraperitoneal, intranasal injections.

One may administer the pharmaceutical composition in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra- cisterna magna injection, intra-parenchymal injection, or a combination thereof. In some embodiments, a pharmaceutical composition disclosed herein can administered to the human patient via at least two administration routes. In some examples, the combination of administration routes by be intracerebroventricular injection and intravenous injection; intrathecal injection and intravenous injection; intra-cisterna magna injection and intravenous injection; and intra-parenchymal injection and intravenous injection.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

Pharmaceutical compositions suitable for use in context of the present disclosure include compositions wherein the active ingredients are contained in an amount effective to achieve the intended purpose. In some embodiments, a therapeutically effective amount means an amount of active ingredients (i.e., modulators and/or inhibitors of Wdr37 disclosed herein) effective to prevent, slow, alleviate or ameliorate symptoms of a disorder (e.g., lymphoproliferative disorders, lymphoid malignancy) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to brain or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. Effective doses may be extrapolated from dose-responsive curves derived from in vitro or in vivo test systems

### III. Methods of Use

In various embodiments, a method for treating Duschenne muscular dystrophy (DMD) in a subject in need thereof is provided, the method comprising administering the gene vector or construct encoding for the Cas9 and sgRNA described above to the subject.

In various embodiments, the gene vector or construct is packaged in an AAV vector.

In still further embodiments, the gene vector or construct is administered systemically (e.g., parenterally). In some embodiments, the gene vector or construct is administered via intravenous injection.

In various embodiments, treating DMD comprises restoring or increasing dystrophin expression in a cell or tissue of the subject. In further embodiments, treating DMD can comprise increasing muscle tone or muscle strength in a tissue of the subject.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### IV. Examples

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples

Duchenne muscular dystrophy (DMD) is a lethal muscle disorder caused by mutations in the DMD gene residing on the X chromosome (Hoffman et al., 1987)(Koenig et al., 1987). The DMD gene encodes the dystrophin protein, which is a large cytoskeletal protein essential for tethering the intracellular actin cytoskeleton and extracellular laminin (Gao and McNally, 2015)(Guiraud et al., 2015). Absence of dystrophin protein in striated muscles causes skeletal muscle degeneration and myocardial fibrosis, and ultimately progresses to fatal respiratory and cardiac failure. With no transformative treatment available, there is an urgent need to develop new therapeutic approaches for DMD.

Genome editing by clustered regularly interspaced short palindromic repeats (CRISPR) and CRISPR-associated proteins (CRISPR-Cas) represents a promising technology to correct disease-causing mutations in the genome (Cong et al., 2013)(Jinek et al., 2012)(Mali et al., 2013). With this approach, Cas9 nuclease is directed by a sequence-specific single guide RNA (sgRNA) to the genome where it can induce double-stranded breaks (DSBs). In the absence of a repair template, DNA DSBs are repaired by two distinct repair pathways, which are non-homologous end joining (NHEJ) when there is no sequence microhomology present at the breakage point, or microhomology-mediated end joining (MMEJ) when there are 2-25 base pairs (bp) of microhomology on each side of the DSB (Iyer et al., 2019)(Gallagher and Haber, 2018).

Recent studies by our group and others explored the potential of CRISPR-Cas9 gene editing and the NHEJ DNA repair pathway as a means of correcting diverse DMD mutations in vivo (Amoasii et al., 2018)(Amoasii et al., 2017)(Bengtsson et al., 2017)(Hakim et al., 2018)(Long et al., 2016)(Min et al., 2020)(Min et al., 2019)(Nelson et al., 2016)(Nelson et al., 2019)(Tabebordbar et al., 2016)(Zhang et al., 2020)(Amoasii et al., 2019). In mice, sustained dystrophin expression and functional improvement can be observed for at least 12-18 months after systemic delivery of CRISPR-Cas9 genome editing components by AAV (Hakim et al., 2018)(Nelson et al., 2019). Nevertheless, challenges remain for therapeutic adaptation of CRISPR-Cas9-mediated gene editing for correction of DMD. For example, the limited packaging capacity of AAV requires a dual system consisting of two AAV vectors to separately package Streptococcus pyogenes Cas9 (SpCas9) and sgRNA. In contrast to SpCas9, the Cas9 ortholog from Staphylococcus aureus (SaCas9) is small enough to be co-packaged with sgRNA into a single AAV vector. However, all current SaCas9-based genome editing systems have used a pair of sgRNAs to induce two DNA DSBs flanking the mutated dystrophin exon (Bengtsson et al., 2017)(Hakim et al., 2018)(Nelson et al., 2016)(Nelson et al., 2019)(Tabebordbar et al., 2016). This “double cut” strategy has been reported to introduce additional unwanted genomic modifications, including inversions and AAV integration (Nelson et al., 2019) (Kwon et al., 2020). Moreover, if one DNA DSB is rejoined by NHEJ repair before the initiation of the second DNA DSB, the mutant exon cannot be excised, rendering this “double cut” strategy ineffective.

In this study, we explored the potential of CRISPR-KKH SaCas9-mediated “single cut” gene editing as a means of correcting an exon 50 DMD deletion mutation. KKH SaCas9 is a SaCas9 variant carrying three amino acid substitutions in the protospacer adjacent motif (PAM)-interacting domain that enable strong genome editing activities at target sites with a 5′-NNNRRT-3′ PAM (Kleinstiver et al., 2015). We first performed “single cut” gene editing with KKH SaCas9 in cardiomyocytes derived from human DMD induced pluripotent stem cells (iPSCs) harboring a deletion of exons 48-50 (ΔEx48-50), the most common “hotspot” region for DMD exon deletions. High frequency of a two-nucleotide deletion was observed after KKH SaCas9-mediated “single cut” gene editing, which restored the open reading frame (ORF) of the dystrophin gene. Next, we packaged KKH SaCas9 and sgRNA into a single AAV9 vector, and performed in vivo genome editing of exon 51 in mice with a deletion of Dmd exon 50. Systemic delivery of the consolidated CRISPR-KKH SaCas9 AAV9 vector showed efficient restoration of dystrophin expression in skeletal muscle and heart and improved muscle contractility. These findings show that delivery of KKH-SaCas9 with a single sgRNA in a single vector system is effective in correcting DMD in vivo, representing an important advancement toward potential therapeutic translation.

Materials and Methods. The following experimental materials and methods were used in the Examples described herein.

Study Design. This study was designed with the primary aim of investigating the feasibility of using CRISPR/SaCas9-mediated “single cut” gene editing for the correction of DMD mutations. The secondary objective was to design an all-in-one AAV packaging system to deliver CRISPR/SaCas9 and sgRNAs for in vivo therapeutic gene editing. We did not use exclusion, randomization, or blind approaches to assign the animals for the experiments. Grip strength tests, histology validation, immunostaining analysis, CK analysis, and muscle electrophysiology were performed as blinded experiments. For each experiment, sample size reflects the number of independent biological replicates and was provided in the figure legends.

KKH SaCas9 Vector Cloning and AAV Vector Production. WT SaCas9 complementary DNA (cDNA) was cut from pX601 plasmid (Ran et al., 2015), a gift from F. Zhang (Addgene plasmid #61591), using Agel-HF and BamHI-HF, and subcloned into pLbCpf1-2A-GFP plasmid by replacing LbCpf1 (Zhang et al., 2017), generating the pSaCas9-2A-GFP plasmid. Modified SaCas9 sgRNA scaffold and KKH SaCas9 C-terminus cDNA (E782K/N968K/R1015H) were synthesized as gBlocks (Integrated DNA Technologies), and subcloned into pSaCas9-2A-GFP plasmid using In-Fusion Cloning Kit (Takara Bio), generating the pKKH-SaCas9-2A-GFP plasmid. The sgRNAs targeting human DMD exon 51 or mouse Dmd exon 51 were subcloned into the newly generated pKKH-SaCas9-2A-GFP plasmid using Bbsl digestion and T4 ligation. KKH SaCas9, 7SK and U6 sgRNA expression cassettes were subcloned into the pSSV9 single-stranded AAV plasmid using In-Fusion Cloning Kit (Takara Bio). Cloning primer sequences are listed in Table 1. AAV viral plasmid was column purified and digested with Smal and Ahdl to check ITR integrity. AAV was packaged by Boston Children's Hospital Viral Core and serotype 9 was chosen for capsid assembly. AAV titer was determined by quantitative real-time PCR assay.

Human iPSC Maintenance, Nucleofection, and Differentiation. DMD ΔEx48-50 iPSCs (RBRC-HPS0164) were purchased from Cell Bank RIKEN BioResource Center. Human iPSCs were cultured in mTeSR plus medium (STEMCELL Technologies) and passaged approximately every 4 days (1:18 split ratio). One hour before nucleofection, iPSCs were treated with 10 μM ROCK inhibitor (Y-27632) and dissociated into single cells using Accutase (Innovative Cell Technologies Inc.). iPSCs (1×10⁶) were mixed with 5 μg of the pKKH-SaCas9-2A-GFP plasmid. The P3 Primary Cell 4D-Nucleofector X Kit (Lonza) was used for nucleofection according to the manufacturer's protocol. After nucleofection, iPSCs were cultured in mTeSR plus medium supplemented with 10 μM ROCK inhibitor, and Primocin (100 μg/ml; InvivoGen). Three days after nucleofection, GFP(+) cells were sorted by FACS and subjected to TIDE analysis. KKH SaCas9-edited iPSC mixtures and single clones were differentiated into cardiomyocytes, as previously described (Min et al., 2020).

Calcium imaging. Calcium imaging was performed as previously described (Atmanli et al., 2019). iPSC-derived cardiomyocytes were replated on glass surfaces at single-cell density and loaded with the fluorescent calcium indicator Fluo-4 AM (Thermo Fisher) at 2 μM. Spontaneous calcium transients of beating iPSC-derived cardiomyocytes were imaged at 37° C. using a Nikon A1R+ confocal system. Calcium transients were processed using Fiji software, and analyzed using Microsoft Excel and Clampfit 10.7 software (Axon Instrument). The calcium release phase was represented with time to peak, which was calculated as the time from baseline to maximal point of the transient. The calcium reuptake phase was represented with the time constant tau by fitting the decay phase of calcium transients with a first-order exponential function.

in vivo AAV Delivery into ΔEx50 Mice. The ΔEx50 DMD mouse model was developed by deleting the mouse Dmd exon 50 using CRISPR/Cas9-mediated mutagenesis (Amoasii et al., 2017). Postnatal day 4 ΔEx50 mice were injected intraperitoneally with 80 μl of AAV9 containing 2×10¹⁴ (low dose) or 4×10¹⁴ vg/kg (high dose) of all-in-one AAV9-KKH-SaCas9-sgRNAs using an ultrafine BD insulin syringe (Becton Dickinson). Four weeks after systemic delivery, ΔEx50 mice and WT littermates were dissected for physiological, biochemical and histological analysis. Animal work described in this manuscript has been approved and conducted under the oversight of the University of Texas Southwestern Institutional Animal Care and Use Committee.

Genomic DNA and RNA Isolation, cDNA Synthesis, and PCR Amplification. Genomic DNA of DMD ΔEx48-50 iPSCs, skeletal muscles and hearts of ΔEx50 mice was isolated using DirectPCR (cell) lysis reagent (Viagen Biotech) according to the manufacturer's protocol. Total RNA of skeletal muscles and heart of ΔEx50 mice was isolated using miRNeasy (QIAGEN) according to the manufacturer's protocol. cDNA was reverse-transcribed from total RNA using iScript Reverse Transcription Supermix (Bio-Rad Laboratories) according to the manufacturer's protocol. Genomic DNA and cDNA was PCR amplified using LongAmp Taq DNA Polymerase (New England BioLabs) PCR products were sequenced and analyzed by TIDE analysis (Brinkman et al., 2014). Primer sequences are listed in Table 1.

Amplicon Deep Sequencing Analysis of Genomic DNA. PCR of genomic DNA was performed using primers designed against the human DMD exon 51, off-target sites, and mouse Dmd exon 51. A second round of PCR was performed to add Illumina flow cell binding sequence and barcodes. All primer sequences are listed in Table 1. Deep sequencing and data analysis were performed as previously described (Amoasii et al., 2017).

Dystrophin Immunocytochemistry and Immunohistochemistry. Dystrophin immunocytochemistry was performed as previously described (Zhang et al., 2017). Primary antibodies used in immunocytochemistry were mouse anti-dystrophin antibody (MANDYS8, Sigma-Aldrich, D8168), rabbit anti-troponin I antibody (H170, Santa Cruz Biotechnology). Secondary antibodies used in immunocytochemistry were biotinylated horse anti-mouse IgG (BMK-2202, Vector Laboratories) and fluorescein-conjugated donkey anti-rabbit IgG (Jackson ImmunoResearch). Skeletal muscles and heart were cryosectioned into eight-micron transverse sections. Immunohistochemistry was performed as previously described (Min et al., 2019). Antibodies used in immunohistochemistry were mouse anti-dystrophin antibody (MANDYS8, Sigma-Aldrich, D8168) and Mouse on Mouse biotinylated anti-mouse IgG (BMK-2202, Vector Laboratories).

Dystrophin Western Blot. For Western blot of iPSC-derived cardiomyocytes, 4×10⁶ cells were lysed in lysis buffer [10% SDS, 62.5 mM tris (pH 6.8), 1 mM EDTA, and protease inhibitor]. Heart and skeletal muscles were crushed into fine powder using a liquid-nitrogen-frozen crushing apparatus. and lysed in the same lysis buffer as iPSC-derived cardiomyocytes. A total 50 μg of protein was loaded onto 4-20% Criterion™ TGX™ Precast Midi Protein Gel (Bio-Rad Laboratories). Details of Western blot running, transferring, and developing were previously described (Min et al., 2020). Primary antibodies used in Western blot were mouse anti-dystrophin antibody (MANDYS8, Sigma-Aldrich, D8168), mouse anti-vinculin antibody (Sigma-Aldrich, V9131). Secondary antibodies used in Western blot were goat anti-mouse horseradish peroxidase (HRP) antibody (Bio-Rad Laboratories).

Electrophysiological Analysis of Isolated EDL and Soleus Muscles. Four weeks after systemic all-in-one AAV9-KKH-SaCas9-sgRNAs gene editing, soleus and EDL muscles of ΔEx50 mice and WT littermates were isolated for electrophysiological analysis. Briefly, soleus and EDL muscles were surgically isolated from 4-week-old ΔEx50 mice, mounted on Grass FT03.C force transducers, bathed in physiological salt solution at 37° C., and gassed continuously with 95% O2-5% CO2. After calibration, muscles were adjusted to initial length at which the passive force was 0.5 g and then stimulated with two platinum wire electrodes to establish optimal length (Lo) for obtaining maximal isometric tetanic tension step by step following the protocol (at 150 Hz for 2 s). Specific force (mN/mm²) was calculated by normalizing contraction force to muscle cross-sectional area.

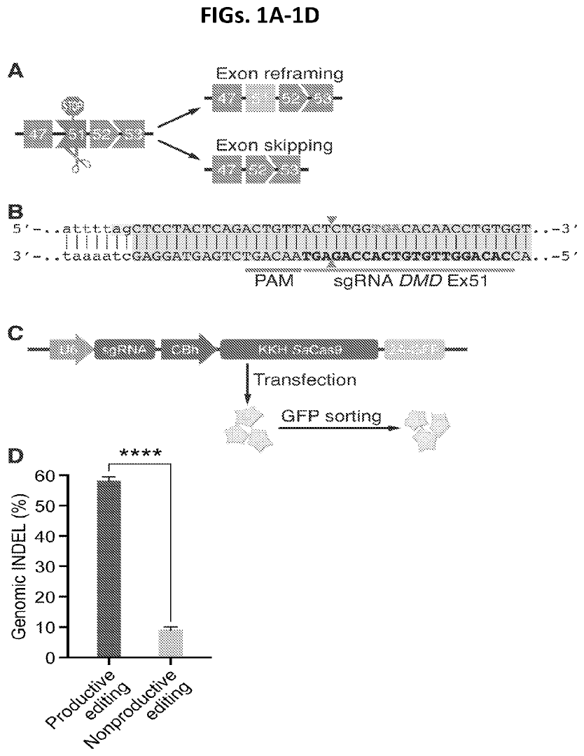

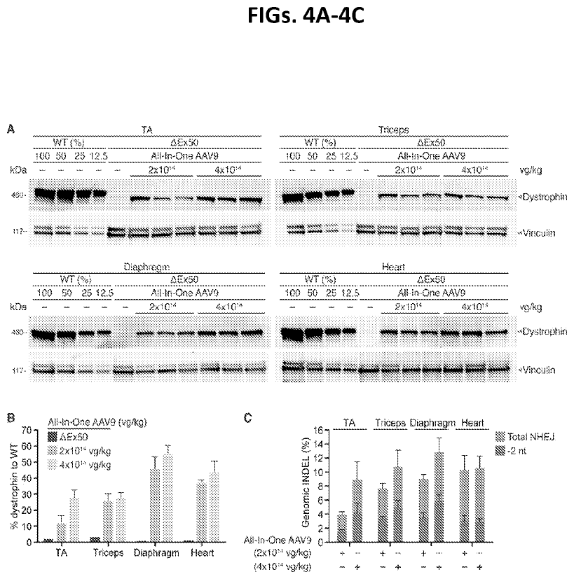

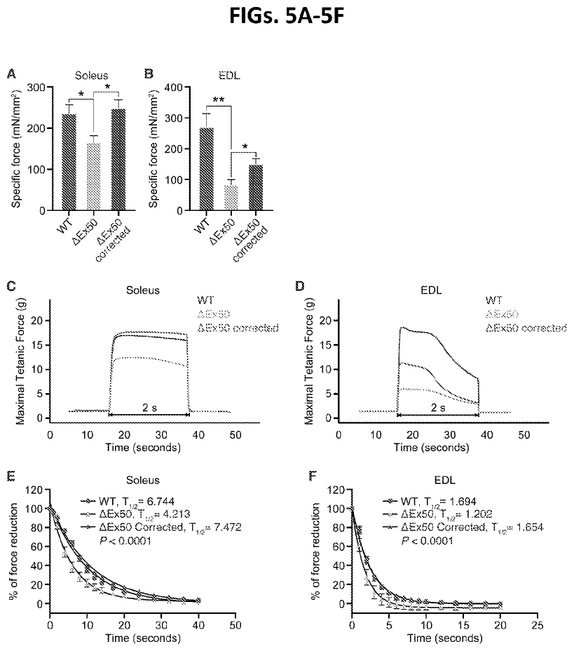

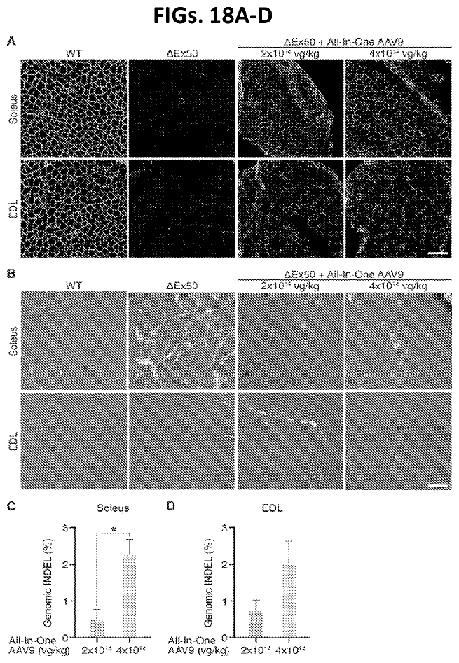

Statistics All data are shown as means ±SEM. Unpaired two-tailed Student's t tests was performed to analyze FIG. 1D, FIG. 18C and 18D; Two-way analysis of variance (ANOVA) with post-hoc Tukey's multiple comparisons test was performed to analyze FIG. 4B; Brown-Forsythe and Welch ANOVA test was performed to analyze FIGS. 5A and 5B;

Nonlinear Regression with Extra sum-of-squares F Test was performed to analyze FIGS. 5E and 5F; One-way ANOVA with post-hoc Tukey's multiple comparisons test was performed to analyze the rest of data. A P<0.05 value was considered statistically significant. Data analyses were performed with GraphPad Prism Software

### Example 1: Strategies for CRISPR-KKH SaCas9-Mediated Genome Editing of Human DMD Exon 51

The majority of DMD deletion mutations are clustered in hotspot regions, comprised of exons 2-20 and exons 45-55, that disrupt the continuity of the ORF with downstream exons. Exon deletions immediately preceding exon 51, which disrupt the reading frame of this exon, represent the most common type of human DMD mutation (Aartsma-Rus et al., 2009). Our ultimate goal was to develop a consolidated AAV expression system encoding SaCas9 and an optimal sgRNA for reframing of exon 51 so as to enable in vivo DMD correction with an All-in-One vector. To optimize this gene editing strategy, we used induced pluripotent stem cells (iPSCs) generated from DMD patients harboring a deletion of exons 48 to 50 (ΔEx48-50) in the DMD gene. This deletion results in splicing of exon 47 to exon 51, which introduces a premature stop codon in exon 51 (FIG. 1A).

We did not identify efficient sgRNAs for WT SaCas9 capable of reframing human DMD exon 51, so we considered various SaCas9 mutants with amino acid substitutions in the PAM-interacting domain that expand the range of DNA cutting by relaxing PAM specificity. From this analysis, we identified a sgRNA for the KKH variant of SaCas9 that recognizes a 5′-AACAGT-3′ PAM in exon 51 and generates a DNA DSB 4-bp upstream of the premature termination codon (FIG. 1B). Depending on the repair outcome, two types of insertions and deletions (INDELs) could restore the exon 51 ORF. Exon 51 could potentially be reframed through INDELs that delete two nucleotides (3n−2), or exon 51 could be skipped if the INDEL is large enough to delete the 5′-AG-3′ splice acceptor (FIG. 1A).

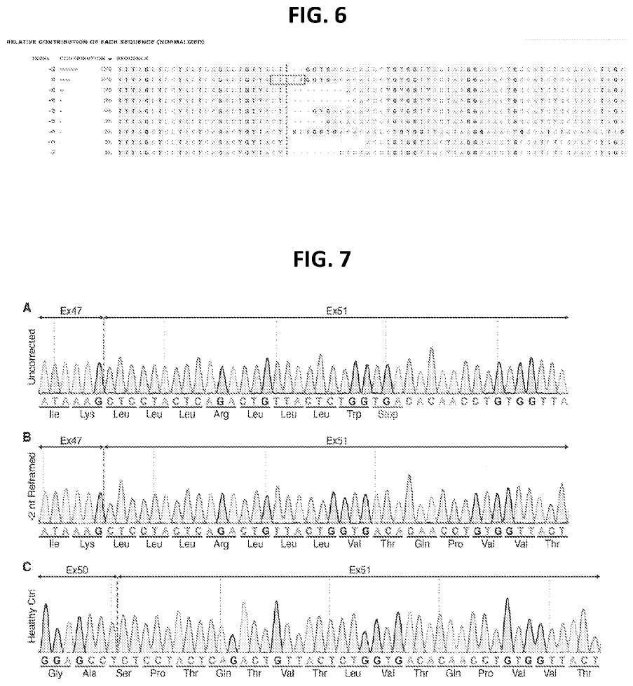

The gene editing efficiency of KKH SaCas9 was tested by transfecting DMD ΔEx48-50 iPSCs with a plasmid expressing KKH SaCas9 and sgRNA, and gene edited cells were enriched through fluorescent activated cell sorting (FACS) (FIG. 1C). We performed tracking of INDELs by decomposition (TIDE) analysis to assess sgRNA cutting efficiency and INDEL patterns. We found that this sgRNA enabled high editing activity of DMD exon 51, generating over 65% of total INDELs (FIG. 1D). More than 55% of INDELs allowed productive editing (3n−2), capable of restoring the DMD exon 51 ORF (FIG. 1D and FIG. 6).

Interestingly, 45% of KKH SaCas9-induced INDELs had a deletion of a 5′-CT-3′ dinucleotide, which allows reframing of the DMD exon 51 ORF (FIG. 6). The sgRNA designed in this study enables the KKH SaCas9 nuclease to induce a DNA DSB between the 5′-CTCT-3′ tetranucleotide, generating a 2 nt 5′-CT-3′ microhomology on each side of the breakage site (FIG. 6), leading to high frequency of precise deletion of the 5′-CT-3′ dinucleotide. These data demonstrate that KKH SaCas9-mediated “single cut” gene editing is an efficient and practicable strategy to restore the dystrophin ORF in DMD exon 51, caused by deletion of preceding exons.

### Example 2: CRISPR-KKH SaCas9-Mediated “Single Cut” Gene Editing Restores Dystrophin Expression in DMD ΔEx48-50 iPSC-Derived Cardiomyocytes

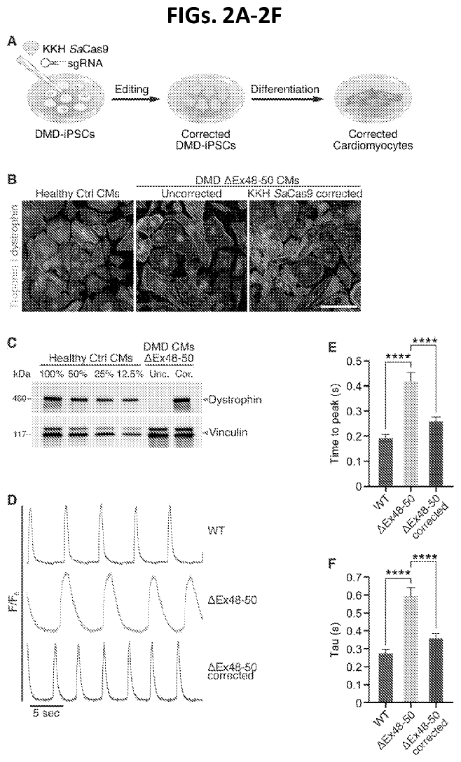

Human iPSCs generated from a ΔEx48-50 DMD patient were corrected by KKH SaCas9 and sgRNA using the “single cut” gene editing approach and then differentiated to cardiomyocytes (iPSC-CMs) (FIG. 2A). The mutation in uncorrected DMD ΔEx48-50 iPSC-CMs results in a premature termination codon following the first eight amino acids encoded by exon 51 (FIG. 7). Correction of DMD ΔEx48-50 iPSC-CMs was accomplished by reframing of the DMD gene, as assessed by reverse transcription PCR (RT-PCR) and sequencing using a forward primer targeting exon 47 and a reverse primer targeting exon 52. Corrected DMD ΔEx48-50 iPSC-CMs had a deletion of the 5′-CT-3′ dinucleotide, which reframed exon 51 (FIG. 7). We confirmed restoration of dystrophin protein expression by immunocytochemistry and Western blot analysis (FIG. 2B and FIG. 2C). Even without clonal selection and expansion, cardiomyocytes differentiated from KKH SaCas9-edited DMD ΔEx48-50 iPSC mixtures showed a high level of dystrophin protein comparable to healthy control iPSC-CMs (FIG. 2B and FIG. 2C).

Dysregulation of calcium handling is a common pathogenic phenotype seen in DMD cardiomyocytes. To assess the consequences of the DMD ΔEx48-50 mutation and the effect of gene editing by the KKH SaCas9-mediated “single cut” strategy, we analyzed spontaneous calcium activity in healthy control and DMD ΔEx48-50 iPSC-CMs (FIG. 2D). Calcium transient kinetics, including time to peak and decay rate, were abnormally elevated in uncorrected DMD ΔEx48-50 iPSC-CMs (FIG. 2E and FIG. 2F). After KKH SaCas9 gene editing, DMD ΔEx48-50 iPSC-CMs displayed normal calcium transient kinetics similar to healthy control iPSC-CMs (FIG. 2E and FIG. 2F), indicating restoration of calcium release and reuptake. Next, we performed genotoxicity analysis in KKH SaCas9-edited DMD ΔEx48-50 iPSC-CMs. We did not observe significant genomic editing at the top eight predicted off-target sites (FIG. 8A-B). Therefore, KKH SaCas9- mediated “single cut” gene editing represents an efficient and safe strategy to restore the ORF of human DMD exon 51 caused by exon 50 deletion, thereby allowing functional restoration in gene edited DMD iPSC-CMs.

### Example 3: Systemic Delivery of All-In-One AAV-Packaged CRISPR-KKH SaCas9 Restores Dystrophin Expression in ΔEx50 Mice

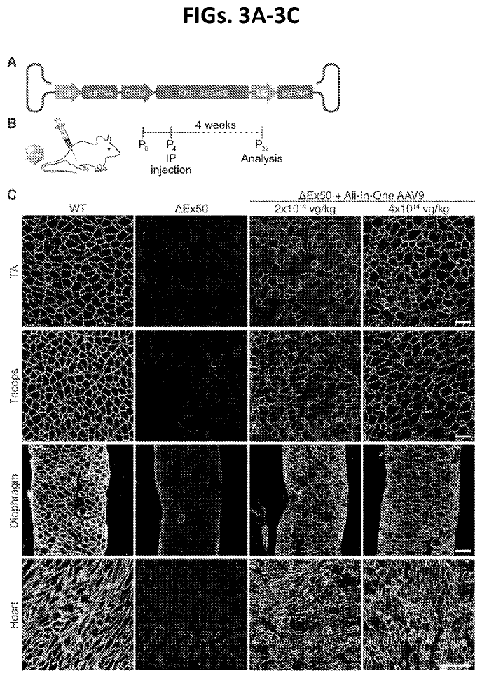

To further evaluate the efficacy of CRISPR-KKH SaCas9 gene editing in vivo, we packaged the KKH SaCas9 nuclease and its sgRNA in one AAV vector (FIG. 3A). In this All-In-One AAV system, KKH SaCas9 expression was driven by a muscle-specific CK8 promoter, restricting its expression to skeletal muscles and heart (Himeda et al., 2011). Because the sgRNA is rate-limiting for in vivo gene editing of DMD mouse models (Hakim et al., 2018)(Min et al., 2019), we included two copies of an expression cassette encoding the same sgRNA (targeting mouse Dmd exon 51) driven by two RNA polymerase III promoters, 7SK and U6, in this All-In-One AAV system (FIG. 3A).

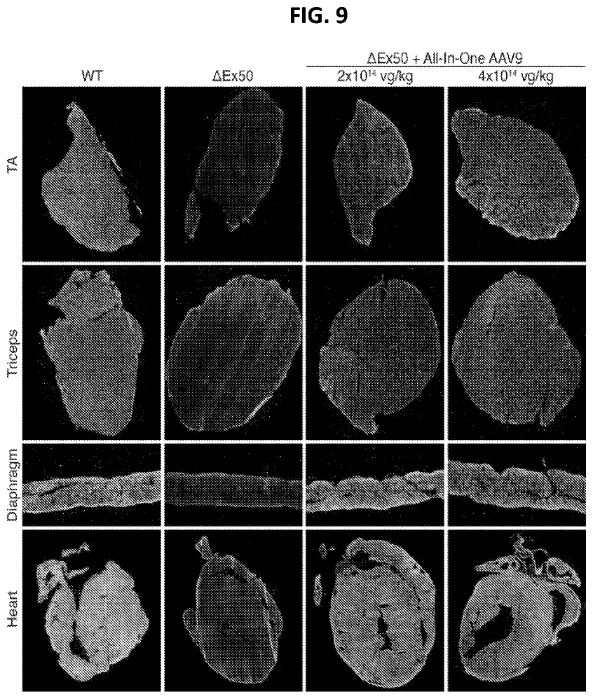

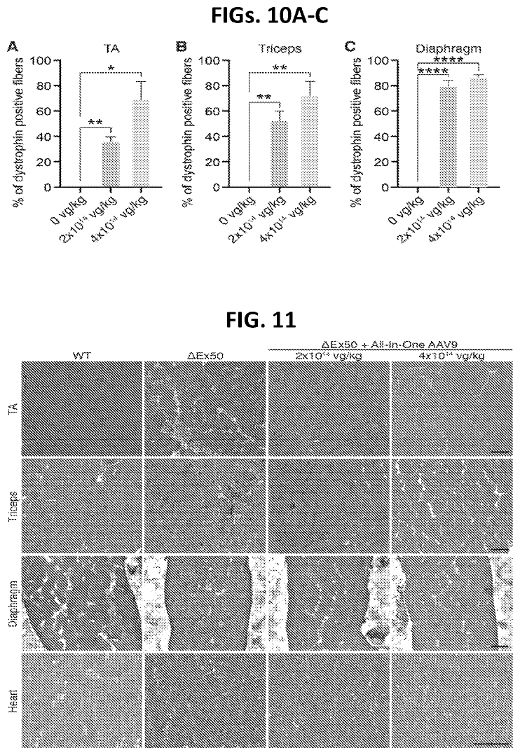

Postnatal day 4 (P4) DMD mice with exon 50 deletion (ΔEx50) were injected intraperitoneally (IP) with All-In-One AAV-packaged KKH SaCas9 at two different doses, 2×10¹⁴ vector genomes (vg)/kg (low dose) and 4×10¹⁴ vg/kg (high dose) (FIG. 3B). Four weeks after systemic AAV delivery, the skeletal muscles and heart of KKH SaCas9-edited ΔEx50 mice were harvested for analysis. Assessment by immunohistochemistry showed that dystrophin restoration in skeletal muscles was dose-dependent (FIG. 3C, FIG. 9, and FIG. 10). ΔEx50 mice receiving the low dose All-In-One AAV treatment displayed 36% and 52% dystrophin-positive myofibers in tibialis anterior (TA) and triceps muscles, respectively (FIG. 10). With low dose All-In-One AAV treatment, the diaphragm showed higher percentages of dystrophin-positive myofibers, reaching 79% (FIG. 10). When the dose of All-In-One AAV was increased to 4×10¹⁴ vg/kg, a substantial increase of dystrophin-positive myofibers in TA and triceps was observed (FIG. 10).

Next, we performed Western blot analysis to quantitatively assess dystrophin restoration in skeletal muscles and heart after systematic delivery of All-In-One AAV-packaged KKH SaCas9 and sgRNA. ΔEx50 mice receiving the low dose All-In-One AAV restored 12% and 26% of dystrophin protein in TA and triceps, respectively (FIG. 4A and FIG. 4B). When the dose of All-In-One AAV was increased to 4×10¹⁴ vg/kg, dystrophin protein restoration in TA and triceps was over 27%. Dystrophin protein expression in the diaphragm and heart exceeded 45% and 38%, respectively, even at the low dose of All-In-One AAV treatment (FIG. 4A and FIG. 4B), indicating that dystrophin protein restoration in the diaphragm and heart is greater than in TA and triceps.

To quantify in vivo gene editing efficiency in ΔEx50 mice, we performed deep sequencing analysis to determine the INDEL frequency and pattern at the genomic level. ΔEx50 mice treated with low dose All-In-One AAV had an average of 4-10% of total INDELs in skeletal muscles and heart; the total INDELs in the high dose group increased to 8-12% (FIG. 4C). Notably, a −2 nt deletion, which is capable of reframing the Dmd exon 51 ORF, was the predominant INDEL in the All-In-One AAV treated ΔEx50 mice. These findings indicate that KKH SaCas9-mediated “single cut” gene editing coupled with the single vector delivery system can effectively correct DMD mutations in vivo.

### Example 4: Systemic Delivery of All-In-One AAV-Packaged CRISPR-KKH SaCas9 Restores Muscle Integrity and Improves Muscle Function in ΔEx50 Mice

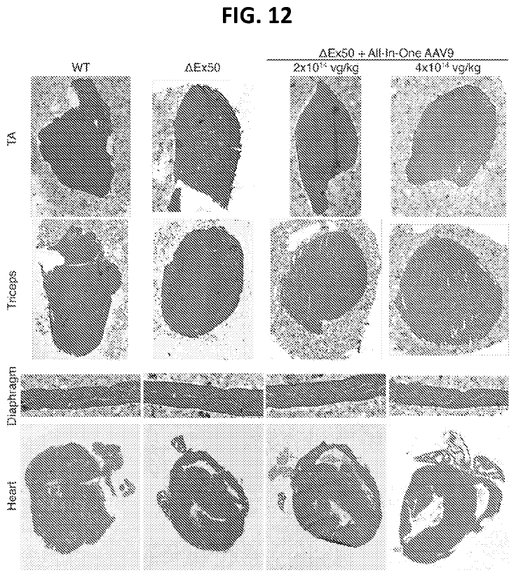

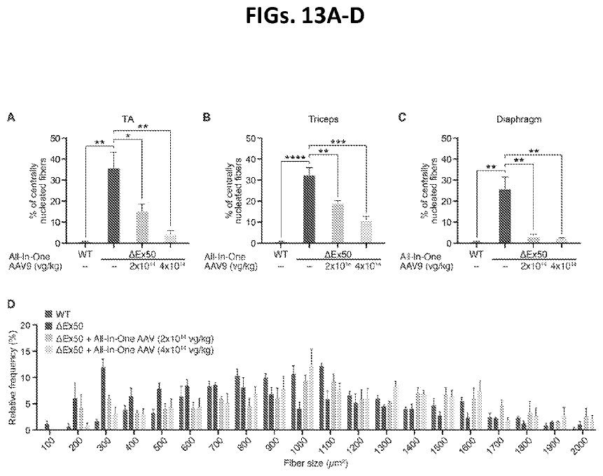

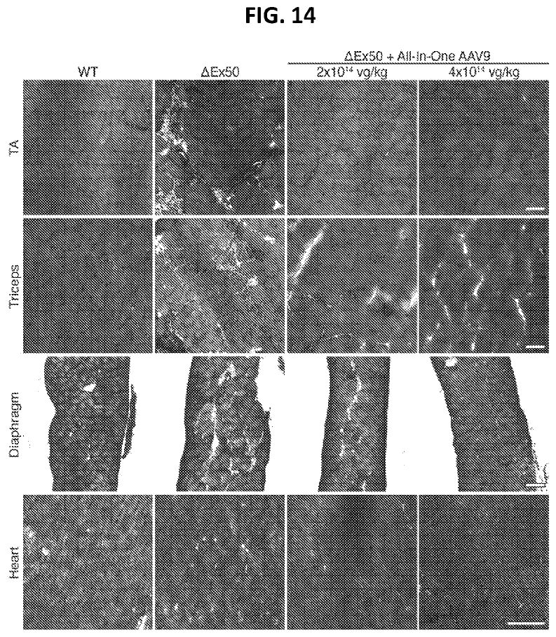

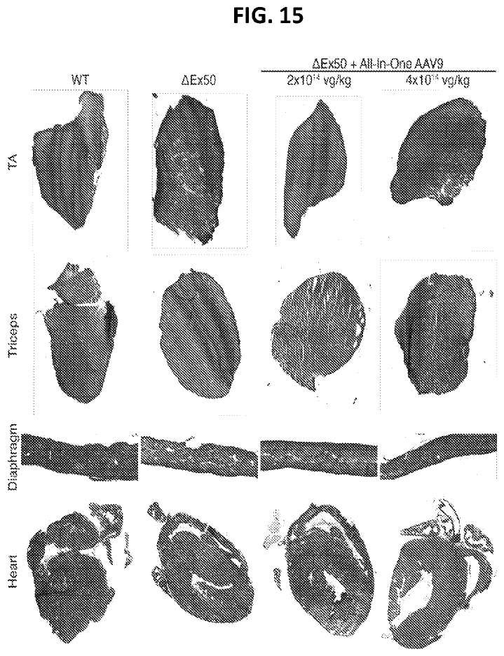

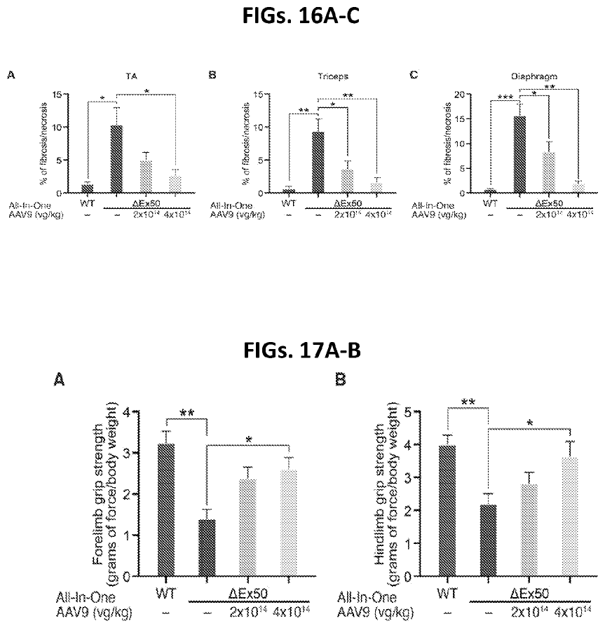

To evaluate whether systemic delivery of All-In-One AAV-packaged KKH SaCas9 was able to rescue pathological phenotypes seen in dystrophic mice, we performed hematoxylin and eosin (H&E) (FIG. 11 and FIG. 12) and Masson's trichrome staining (FIG. 14 and FIG. 15) of skeletal muscles and heart isolated from ΔEx50 mice four weeks after KKH SaCas9-mediated gene editing. Skeletal muscles from ΔEx50 mice without gene editing displayed necrosis and inflammatory infiltration (FIG. 11 and FIG. 12). The percentage of regenerating myofibers with central nuclei in untreated ΔEx50 mice was between 25-35% across different skeletal muscle groups (FIGS. 13A-C). After All-In- One AAV treatment, the percentage of centrally nucleated myofibers declined substantially (FIGS. 13A-C). Distribution of myofiber cross-sectional area also showed an improvement in the TA muscle after delivery of All-In-One AAV at both doses (FIG. 13D). Masson's trichrome staining showed substantial fibrosis and necrosis in untreated ΔEx50 mice (FIG. 14 and FIG. 15), ranging between 10-15% across different skeletal muscle groups (FIGS. 16A-C). After All-In-One AAV treatment, the percentage of fibrotic and necrotic area dramatically declined (FIG. 14 to FIG. 16).

To examine the effect of gene editing on muscle function, we performed grip strength analysis on ΔEx50 mice at four weeks after systemic All-In-One AAV delivery. ΔEx50 mice without gene editing showed a 56% and 45% reduction of grip strength in forelimb and hindlimb compared to the WT littermates, respectively (FIG. 17). Forelimb and hindlimb grip strength of ΔEx50 mice receiving low dose All-In-One AAV treatment showed a trend toward improvement (FIG. 17). Moreover, ΔEx50 mice receiving high dose All-In-One AAV treatment showed a dramatic improvement of forelimb and hindlimb grip strength by 86% and 67%, respectively, compared to the untreated ΔEx50 littermates (FIG. 17). In addition, we also performed electrophysiological analysis on soleus and extensor digitorum longus (EDL) muscles isolated from ΔEx50 mice at four weeks after receiving the high dose All-In-One AAV treatment. We observed rescue of specific force and maximal tetanic force in the soleus and EDL muscle of the corrected ΔEx50 mice (FIG. 5A-FIG. 5D). Without KKH SaCas9 gene editing, muscle force was reduced by 30% in slow-twitch soleus muscle and 69% in fast-twitch EDL muscle compared to the WT littermates (FIG. 5A and FIG. 5B). After systemic delivery of All-In-One AAV-packaged KKH SaCas9, muscle-specific force of the soleus and EDL was increased by 51% and 78%, respectively, compared to the untreated ΔEx50 littermates (FIGS. 5A and 5B). The maximal tetanic force of the soleus and EDL also followed a similar pattern as seen for specific force (FIG. 5C and FIG. 5D).

Next, we performed fatigue analysis in WT and ΔEx50 mice. Without KKH SaCas9 gene editing, ΔEx50 mice exhibited faster force reduction in soleus and EDL. The average time for 50% force reduction in soleus and EDL from ΔEx50 mice was reduced by 38% and 29%, respectively, compared to the WT littermates (FIG. 5E and FIG. 5F). After KKH SaCas9-mediated gene editing, the force reduction rate of soleus and EDL from ΔEx50 mice was restored to the WT level (FIG. 5E and FIG. 5F), indicating enhanced fatigue resistance. Improvement of muscle function correlated with increased dystrophin expression and decreased muscle degeneration (FIG. 18).

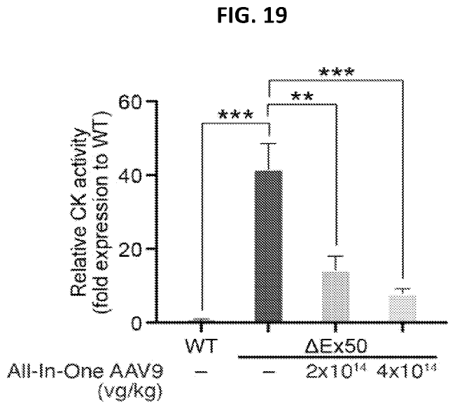

Elevated serum creatine kinase (CK) is a pathological hallmark of DMD. After receiving the low and high dose All-In-One AAV treatment, CK levels in the ΔEx50 mice were reduced by 66% and 81%, respectively, compared to the untreated ΔEx50 littermates (FIG. 19). Together, these findings demonstrate that KKH SaCas9-mediated “single cut” gene editing improves muscle integrity and provides functional benefit to DMD ΔEx50 mice

### Discussion of the Examples

Despite intense efforts to develop therapeutic strategies to restore dystrophin expression in DMD patients through oligonucleotide-mediated exon skipping and gene therapy with truncated forms of dystrophin, there remains a major unmet need for approaches to restore maximal portions of the dystrophin gene in patients with different DMD deletions (Chemello et al., 2020)(Min et al., 2019)(Zhang et al., 2018). Exon deletions that disrupt the continuity of the dystrophin ORF in exon 51 represent the most predominant cause of DMD. Skipping or reframing exon 51, in principle, can provide therapeutic benefit to ˜13% of the DMD population (Aartsma-Rus et al., 2009). To date, there is no report of using SaCas9-mediated “single cut” gene editing to correct DMD mutations. Previously published studies employed two sgRNAs to direct SaCas9 to induce two DNA DSBs flanking an out-of-frame exon (Bengtsson et al., 2017)(Hakim et al., 2018)(Nelson et al., 2016)(Nelson et al., 2019)(Tabebordbar et al., 2016)(Kwon et al., 2020).

In this study, we developed a “single cut” gene editing strategy in which KKH SaCas9 introduces a single DNA DSB within exon 51 to reframe the dystrophin ORF in human cardiomyocytes lacking exons 48-50 and in mouse muscles lacking exon 50. Cardiomyocytes derived from human iPSCs with the ΔEx48-50 mutation and corrected by editing with KKH SaCas9 restored dystrophin expression and showed improved calcium transient kinetics. We also packaged KKH SaCas9 and its sgRNA into a single AAV vector and performed in vivo gene editing. DMD ΔEx50 mice receiving systemic All-In-One AAV treatment restored dystrophin expression with consequent improvement in muscle contractility and force. This study represents the first application of KKH SaCas9-mediated “single cut” gene editing for the treatment of DMD.

SpCas9-mediated “single cut” gene editing has been widely used for correcting diverse DMD mutations with high efficiency, especially for mutations that can be reframed by a 1-bp insertion (Amoasii et al., 2018)(Amoasii et al., 2017)(Min et al., 2020)(Min et al., 2019)(Zhang et al., 2020)(Amoasii et al., 2019). In contrast, prior studies of SaCas9-mediated correction of DMD mutations relied on two sgRNAs to completely excise the out-of-frame exon (Bengtsson et al., 2017)(Hakim et al., 2018)(Nelson et al., 2016)(Nelson et al., 2019)(Tabebordbar et al., 2016)(Kwon et al., 2020). These different approaches are dependent on the topological distinctions between the DNA DSBs induced by Sp- and SaCas9. Studies using molecular dynamics simulations suggest that an SpCas9-induced DNA DSB generates a staggered cut, producing a single nucleotide 5′ overhang, leading to a high frequency of a 1-bp insertion after NHEJ-mediated repair (Zuo and Liu, 2016) (Lemos et al., 2018). In contrast, the DSB generated by SaCas9 cutting is blunt ended, with INDELs with varying lengths. Therefore, NHEJ-mediated 1-bp insertion appears to be an SpCas9-specific phenomenon, which does not apply to SaCas9 (Shen et al., 2018). This distinction poses limitations to SaCas9-mediated “single cut” gene editing as a general strategy for reframing out-of-frame exons. In order to address this issue, we screened for sgRNAs capable of directing KKH SaCas9 to induce a DNA DSB between a microhomology sequence. Studies have demonstrated that DNA DSB around regions of microhomology tend to generate deletions with predictable length (lyer et al., 2019)(Shen et al., 2018). As expected, with SaCas9 cutting, we observed a majority of productive editing events containing a precise deletion of the 5′-CT-3′ dinucleotide.

Although CRISPR correction of DMD has shown promise in pre-clinical studies, several questions and challenges remain to be addressed. The first concern is durability of CRISPR gene editing in muscle cells. Skeletal muscle has resident stem cells (satellite cells) capable of regenerating or fusing to myofibers (Yin et al., 2013). Although there is increasing evidence that AAV9 delivery of CRISPR-Cas9 components can transduce and edit satellite cells, (Tabebordbar et al., 2016)(Kwon et al., 2020) (Nance et al., 2019) the efficiency of viral transduction and gene editing in satellite cells remains low. Whether unedited satellite cells will gradually dilute out corrected nuclei in regenerating myofibers remains unknown. Engineering novel AAV serotypes with strong tropism to satellite cells may offer a potential solution to this issue. Another concern is the AAV dose administered in gene editing. In pre-clinical studies, the average AAV dose used in in vivo gene editing of DMD animal models varies between 1.6×10¹⁴ to 1.8×10¹⁵ vg/kg (Amoasii et al., 2018)(Amoasii et al., 2017)(Bengtsson et al., 2017)(Hakim et al., 2018)(Long et al., 2016)(Min et al., 2019)(Nelson et al., 2016)(Nelson et al., 2019)(Tabebordbar et al., 2016)(Zhang et al., 2020)(Amoasii et al., 2019)(Kwon et al., 2020), which becomes an obvious burden for industrial production and clinical translation. Similarly, AAV dose of 2.0×10¹⁴ vg/kg or higher has been necessary to direct therapeutically beneficial level of micro-dystrophin in early stage clinical trials (Duan, 2018).

Self-complementary AAV has been shown to be superior to single-stranded AAV in viral transduction and CRISPR gene editing (Min et al., 2020)(Zhang et al., 2020). When self-complementary AAV is used for CRISPR sgRNA delivery, the viral dose can be reduced to 8×10¹³ vg/kg. However, SaCas9 used in this study is too large to be packaged into self-complementary AAV. Potential solutions to address these concerns include (i) screening more compact CRISPR/Cas system to bypass the packaging limit of self-complementary AAV, and (ii) dividing SaCas9 into two parts to accommodate self-complementary AAV packaging and using the split-intein system to reconstitute the full-length Cas9 after AAV delivery (Truong et al., 2015)(Chew et al., 2016).

Although complete restoration of normal levels of dystrophin is not achievable for therapeutic gene editing because AAV viral transduction of skeletal muscles is not 100%, studies in patients with Becker muscular dystrophy have estimated that ˜15% of normal levels of dystrophin protein could provide therapeutic benefits (Hoffman et al., 1998). Our in vivo data demonstrate that All-In-One SaCas9-mediated “single cut” gene editing has high efficiency in dystrophin restoration, capable of restoring 30-50% of dystrophin protein levels in multiple skeletal muscles and 50% in the heart within 4 weeks of administration. Therefore, All-In-One SaCas9-mediated “single cut” gene editing developed in this study shows strong potential for therapeutic translation and represents a promising therapy for permanent correction of DMD.

Finally, the inventors have recently reported the effectiveness of base editing as a strategy for exon skipping in DMD via splice site modification (Chemello et al., 2021). Whether this approach might be adapted to an All-In-One strategy is under investigation. Together, these various approaches add to the expanding toolbox of gene editing strategies that may ultimately be applied to different DMD mutations.

TABLE 1

| Primer | | |
|---|---|---|
| function | Primer name | Primer sequence |
| Generating | HindIII-SaCas9-F1 | CCCCGTCGTGAAGAGAAGCTT (SEQ ID NO: 1) |
| pKKH- | R781-SaCas9-R1 | TCTATTAGGCTTCTTGTCCACCCG (SEQ ID NO: 2) |
| SaCas9-2A- | XhoI-Pacl-eCK8-F1 | AGAAGAAATATAAGACTCGAGTTAATTAATGCCCATGTAAGGA |
| GFP and | | GGCAAGG (SEQ ID NO: 3) |
| AAV-CK8- | NcoI-eCK8-R1 | GCTTCTTCTTTGGGGCCATGGTGGCGCTAGCGCTG (SEQ ID |
| KKH- | | NO: 4) |
| SaCas9 | | |
| Human DMD | DMD-Ex51-T7E1-F1 | ATTTCCCTGGCAAGGTCTGAA (SEQ ID NO: 5) |
| Ex51 | DMD-Ex51-T7E1-R1 | ATCCTCAAGGTCACCCACCA (SEQ ID NO: 6) |
| TIDE analysis | | |
| Human DMD | DMD-Ex51-Sa-N21-Top2 | CACCGCACAGGTTGTGTCACCAGAGT (SEQ ID NO: 7) |
| Ex51 | KKH | |
| KKH sgRNA | DMD-Ex51-Sa-N21-Bot2 | AGACACTCTGGTGACACAACCTGTGC (SEQ ID NO: 8) |
| | KKH | |
| Human DMD | DMD-Ex51-RT-PCR-F1 | CCCAGAAGAGCAAGATAAACTTGAA (SEQ ID NO: 9) |
| Ex47- | DMD-Ex51-RT-PCR-R1 | CTCTGTTCCAAATCCTGCATTGT (SEQ ID NO: 10) |
| 52 RT PCR | | |
| Mouse Dmd | Dmd-Ex51-T7E1-F1 | CCTGAAAATTCCCCAATACTTGTGC (SEQ ID NO: 11) |
| Ex51 | Dmd-Ex51-T7E1-R1 | GTGTTTCACCCTAACTTTTCATCCC (SEQ ID NO: 12) |
| TIDE analysis | | |
| Mouse Dmd | Dmd-Ex51-Sa-N21-Top2 | CACCGCACAGATTGTGTCACTAGAGT (SEQ ID NO: 13) |
| Ex51 | KKH | |
| KKH sgRNA | Dmd-Ex51-Sa-N21-Bot2 | AGACACTCTAGTGACACAATCTGTGC (SEQ ID NO: 14) |
| | KKH | |
| Mouse Dmd | KKH-mE51-ON-DS-F1 | TCGTCGGCAGCGTCAGATGTGTATAAGAGACAGCCTGAAAATT |
| Ex51 | | CCCCAATACTTGTGC |
| on-target | | (SEQ ID NO: 15) |
| | KKH-mE51-ON-DS-R1 | GTCTCGTGGGCTCGGAGATGTGTATAAGAGACAGTCCAAGCTC |
| | | GGTTGAAGTCTG |
| | | (SEQ ID NO: 16) |
| Human DMD | KKH-hE51-ON-DS-F1 | TCGTCGGCAGCGTCAGATGTGTATAAGAGACAGTCATGAATAA |
| Ex51 | | GAGTTTGGCTCAAATTG |
| on-target | | (SEQ ID NO: 17) |
| | KKH-hE51-ON-DS-R1 | GTCTCGTGGGCTCGGAGATGTGTATAAGAGACAGGTGGGAAAT |
| | | GGTCTAGGAGAGTAAAG |
| | | (SEQ ID NO: 18) |
| Human DMD | KKH-hE51-OT1-DS-F2 | TCGTCGGCAGCGTCAGATGTGTATAAGAGACAGCATCATTCCA |
| sgRNA off- | | TATCTATCACCTAGCC |
| target | | (SEQ ID NO: 19) |
| | KKH-hE51-OT1-DS-R2 | GTCTCGTGGGCTCGGAGATGTGTATAAGAGACAGCTTCAGAGA |
| | | ATTGACCAACCACTG |
| | | (SEQ ID NO: 20) |
| | KKH-hE51-OT2-DS-F2 | TCGTCGGCAGCGTCAGATGTGTATAAGAGACAGCCAATTCTCT |
| | | GTATCAAATCTTCTTGG |
| | | (SEQ ID NO: 21) |
| | KKH-hE51-OT2-DS-R2 | GTCTCGTGGGCTCGGAGATGTGTATAAGAGACAGGCAGGCTGG |
| | | GAATATCAGGTTC |
| | | (SEQ ID NO: 22) |
| | KKH-hE51-OT3-DS-F1 | TCGTCGGCAGCGTCAGATGTGTATAAGAGACAGGAGTCAGAAT |
| | | TTCACCATGTTAGCC |
| | | (SEQ ID NO: 23) |
| | KKH-hE51-OT3-DS-R1 | GTCTCGTGGGCTCGGAGATGTGTATAAGAGACAGTTCCCTTTA |
| | | ACCCAAGACCCTG |
| | | (SEQ ID NO: 24) |
| | KKH-hE51-OT4-DS-F1 | TCGTCGGCAGCGTCAGATGTGTATAAGAGACAGGTGTGTGGGT |
| | | GGGGAAGAAT |
| | | (SEQ ID NO: 25) |
| | KKH-hE51-OT4-DS-R1 | GTCTCGTGGGCTCGGAGATGTGTATAAGAGACAGTCACAGAGG |
| | | TAGCCTGGACA |
| | | (SEQ ID NO: 26) |
| | KKH-hE51-OT5-DS-F1 | TCGTCGGCAGCGTCAGATGTGTATAAGAGACAGAGCACATGGT |
| | | TATCCTTGACGT |
| | | (SEQ ID NO: 27) |
| | KKH-hE51-OT5-DS-R1 | GTCTCGTGGGCTCGGAGATGTGTATAAGAGACAGCTTTCATTC |
| | | CTTGAATCTGACTTCA |
| | | (SEQ ID NO: 28) |
| | KKH-hE51-OT6-DS-F1 | TCGTCGGCAGCGTCAGATGTGTATAAGAGACAGACGGACTCTC |
| | | AAACTGCTCC |
| | | (SEQ ID NO: 29) |
| | KKH-hE51-OT6-DS-R1 | GTCTCGTGGGCTCGGAGATGTGTATAAGAGACAGGCCCTCATT |
| | | GCTGACCTTCTC |
| | | (SEQ ID NO: 30) |
| | KKH-hE51-OT7-DS-F1 | TCGTCGGCAGCGTCAGATGTGTATAAGAGACAGCGAAGGATTC |
| | | TATTGCCTTTGGTG |
| | | (SEQ ID NO: 31) |
| | KKH-hE51-OT7-DS-R1 | GTCTCGTGGGCTCGGAGATGTGTATAAGAGACAGACTTCTGCT |
| | | TACACCTTCACATAGTG |
| | | (SEQ ID NO: 32) |
| | KKH-hE51-OT8-DS-F1 | TCGTCGGCAGCGTCAGATGTGTATAAGAGACAGCACCTGTGCC |
| | | CACCTTATAGTG |
| | | (SEQ ID NO: 33) |
| | KKH-hE51-OT8-DS-R1 | GTCTCGTGGGCTCGGAGATGTGTATAAGAGACAGCTTTGATTA |
| | | GAGACAAGGGACCCAG |
| | | (SEQ ID NO: 34) |
| | miSeq-Univ-F | AATGATACGGCGACCACCGAGATCTACACTCGTCGGC |
| | | AGCGTC (SEQ ID NO: 35) |
| | miSeq-Barcode-R | CAAGCAGAAGACGGCATACGAGATxxxxxxGTCTCGT |
| | | GGGCTCGG (SEQ ID NO: 36) |

UNDERLINE: Nextera adaptor sequence;
BOLDED: Transposon end sequence
xxxxxx: barcode sequence

TABLE 2
Additional Sequences

| Description | Sequence | SEQ ID NO. |
|---|---|---|
| Exon 51 Spacer | ACTCTGGTGACACAACCTGTG | 37 |
| Sequence | | |
| Exon 51 Target | TGAGACCACTGTGTTGGACAC | 38 |
| Sequence | | |
| saCas9 Scaffold | GTTTAAGTACTCTGTGCTGGAAACAGCACAGAATCTACTTAAACAAG | 39 |
| v2 | GCAAAATGCCGTGTTTATCTCGTCAACTTGTTGGCGAGAT | |
| SaCas9 | KRNYILGLDIGITSVGYGIIDYETRDVIDAGVRLFKEANVENNEGRR | 40 |
| | SKRGARRLKRRRRHRIQRVKKLLFDYNLLTDHSELSGINPYEARVKG | |
| | LSQKLSEEEFSAALLHLAKRRGVHNVNEVEEDTGNELSTKEQISRNS | |
| | KALEEKYVAELQLERLKKDGEVRGSINRFKTSDYVKEAKQLLKVQKA | |
| | YHQLDQSFIDTYIDLLETRRTYYEGPGEGSPFGWKDIKEWYEMLMGH | |
| | CTYFPEELRSVKYAYNADLYNALNDLNNLVITRDENEKLEYYEKFQI | |
| | IENVFKQKKKPTLKQIAKEILVNEEDIKGYRVTSTGKPEFTNLKVYH | |
| | DIKDITARKEIIENAELLDQIAKILTIYQSSEDIQEELTNLNSELTQ | |
| | EEIEQISNLKGYTGTHNLSLKAINLILDELWHINDNQIAIFNRLKLV | |
| | PKKVDLSQQKEIPTTLVDDFILSPVVKRSFIQSIKVINAIIKKYGLP | |
| | NDIIIELAREKNSKDAQKMINEMQKRNRQTNERIEEIIRTTGKENAK | |
| | YLIEKIKLHDMQEGKCLYSLEAIPLEDLLNNPFNYEVDHIIPRSVSF | |
| | DNSFNNKVLVKQEENSKKGNRTPFQYLSSSDSKISYETFKKHILNLA | |
| | KGKGRISKTKKEYLLEERDINRFSVQKDFINRNLVDTRYATRGLMNL | |
| | LRSYFRVNNLDVKVKSINGGFTSFLRRKWKFKKERNKGYKHHAEDAL | |
| | IIANADFIFKEWKKLDKAKKVMENQMFEEKQAESMPEIETEQEYKEI | |
| | FITPHQIKHIKDFKDYKYSHRVDKKPNRELINDTLYSTRKDDKGNTL | |
| | IVNNLNGLYDKDNDKLKKLINKSPEKLLMYHHDPQTYQKLKLIMEQY | |
| | GDEKNPLYKYYEETGNYLTKYSKKDNGPVIKKIKYYGNKLNAHLDIT | |
| | DDYPNSRNKVVKLSLKPYRFDVYLDNGVYKFVTVKNLDVIKKENYYE | |
| | VNSKCYEEAKKLKKISNQAEFIASFYNNDLIKINGELYRVIGVNNDL | |
| | LNRIEVNMIDITYREYLENMNDKRPPRIIKTIASKTQSIKKYSTDIL | |
| | GNLYEVKSKKHPQIIKKG | |
| SaCas9-KKH | KRNYILGLDIGITSVGYGIIDYETRDVIDAGVRLFKEANVENNEGRR | 41 |
| | SKRGARRLKRRRRHRIQRVKKLLFDYNLLTDHSELSGINPYEARVKG | |
| | LSQKLSEEEFSAALLHLAKRRGVHNVNEVEEDTGNELSTKEQISRNS | |
| | KALEEKYVAELQLERLKKDGEVRGSINRFKTSDYVKEAKQLLKVQKA | |
| | YHQLDQSFIDTYIDLLETRRTYYEGPGEGSPFGWKDIKEWYEMLMGH | |
| | CTYFPEELRSVKYAYNADLYNALNDLNNLVITRDENEKLEYYEKFQI | |
| | IENVFKQKKKPTLKQIAKEILVNEEDIKGYRVTSTGKPEFTNLKVYH | |
| | DIKDITARKEIIENAELLDQIAKILTIYQSSEDIQEELTNLNSELTQ | |
| | EEIEQISNLKGYTGTHNLSLKAINLILDELWHINDNQIAIFNRLKLV | |
| | PKKVDLSQQKEIPTTLVDDFILSPVVKRSFIQSIKVINAIIKKYGLP | |
| | NDIIIELAREKNSKDAQKMINEMQKRNRQTNERIEEIIRTTGKENAK | |
| | YLIEKIKLHDMQEGKCLYSLEAIPLEDLLNNPFNYEVDHIIPRSVSF | |
| | DNSFNNKVLVKQEENSKKGNRTPFQYLSSSDSKISYETFKKHILNLA | |
| | KGKGRISKTKKEYLLEERDINRFSVQKDFINRNLVDTRYATRGLMNL | |
| | LRSYFRVNNLDVKVKSINGGFTSFLRRKWKFKKERNKGYKHHAEDAL | |
| | IIANADFIFKEWKKLDKAKKVMENQMFEEKQAESMPEIETEQEYKEI | |
| | FITPHQIKHIKDFKDYKYSHRVDKKPNRKLINDTLYSTRKDDKGNTL | |
| | IVNNLNGLYDKDNDKLKKLINKSPEKLLMYHHDPQTYQKLKLIMEQY | |
| | GDEKNPLYKYYEETGNYLTKYSKKDNGPVIKKIKYYGNKLNAHLDIT | |
| | DDYPNSRNKVVKLSLKPYRFDVYLDNGVYKFVTVKNLDVIKKENYYE | |
| | VNSKCYEEAKKLKKISNQAEFIASFYKNDLIKINGELYRVIGVNNDL | |
| | LNRIEVNMIDITYREYLENMNDKRPPHIIKTIASKTQSIKKYSTDIL | |
| | GNLYEVKSKKHPQIIKKG | |
| SaCas9-HF | KRNYILGLDIGITSVGYGIIDYETRDVIDAGVRLFKEANVENNEGRR | 42 |
| | SKRGARRLKRRRRHRIQRVKKLLFDYNLLTDHSELSGINPYEARVKG | |
| | LSQKLSEEEFSAALLHLAKRRGVHNVNEVEEDTGNELSTKEQISRNS | |
| | KALEEKYVAELQLERLKKDGEVRGSINRFKTSDYVKEAKQLLKVQKA | |
| | YHQLDQSFIDTYIDLLETRRTYYEGPGEGSPFGWKDIKEWYEMLMGH | |
| | CTYFPEELASVKYAYNADLYNALNDLNNLVITRDENEKLEYYEKFQI | |
| | IENVFKQKKKPTLKQIAKEILVNEEDIKGYRVTSTGKPEFTNLKVYH | |
| | DIKDITARKEIIENAELLDQIAKILTIYQSSEDIQEELTNLNSELTQ | |
| | EEIEQISNLKGYTGTHNLSLKAINLILDELWHINDAQIAIFARLKLV | |
| | PKKVDLSQQKEIPTTLVDDFILSPVVKRSFIQSIKVINAIIKKYGLP | |
| | NDIIIELAREKNSKDAQKMINEMQKRNRQTNERIEEIIRTTGKENAK | |
| | YLIEKIKLHDMQEGKCLYSLEAIPLEDLLNNPFNYEVDHIIPRSVSF | |
| | DNSFNNKVLVKQEENSKKGNRTPFQYLSSSDSKISYETFKKHILNLA | |
| | KGKGRISKTKKEYLLEERDINRFSVQKDFINRNLVDTRYATAGLMNL | |
| | LRSYFRVNNLDVKVKSINGGFTSFLRRKWKFKKERNKGYKHHAEDAL | |
| | IIANADFIFKEWKKLDKAKKVMENQMFEEKQAESMPEIETEQEYKEI | |
| | FITPHQIKHIKDFKDYKYSHRVDKKPNRELINDTLYSTRKDDKGNTL | |
| | IVNNLNGLYDKDNDKLKKLINKSPEKLLMYHHDPQTYQKLKLIMEQY | |
| | GDEKNPLYKYYEETGNYLTKYSKKDNGPVIKKIKYYGNKLNAHLDIT | |
| | DDYPNSRNKVVKLSLKPYRFDVYLDNGVYKFVTVKNLDVIKKENYYE | |
| | VNSKCYEEAKKLKKISNQAEFIASFYNNDLIKINGELYRVIGVNNDL | |
| | LNRIEVNMIDITYREYLENMNDKRPPRIIKTIASKTQSIKKYSTDIL | |
| | GNLYEVKSKKHPQIIKKG | |
| SaCas9-KKH-HF | KRNYILGLDIGITSVGYGIIDYETRDVIDAGVRLFKEANVENNEGRR | 43 |
| | SKRGARRLKRRRRHRIQRVKKLLFDYNLLTDHSELSGINPYEARVKG | |
| | LSQKLSEEEFSAALLHLAKRRGVHNVNEVEEDTGNELSTKEQISRNS | |
| | KALEEKYVAELQLERLKKDGEVRGSINRFKTSDYVKEAKQLLKVQKA | |
| | YHQLDQSFIDTYIDLLETRRTYYEGPGEGSPFGWKDIKEWYEMLMGH | |
| | CTYFPEELASVKYAYNADLYNALNDLNNLVITRDENEKLEYYEKFQI | |
| | IENVFKQKKKPTLKQIAKEILVNEEDIKGYRVTSTGKPEFTNLKVYH | |
| | DIKDITARKEIIENAELLDQIAKILTIYQSSEDIQEELTNLNSELTQ | |
| | EEIEQISNLKGYTGTHNLSLKAINLILDELWHTNDAQIAIFARLKLV | |
| | PKKVDLSQQKEIPTTLVDDFILSPVVKRSFIQSIKVINAIIKKYGLP | |
| | NDIIIELAREKNSKDAQKMINEMQKRNRQTNERIEEIIRTTGKENAK | |
| | YLIEKIKLHDMQEGKCLYSLEAIPLEDLLNNPFNYEVDHIIPRSVSF | |
| | DNSFNNKVLVKQEENSKKGNRTPFQYLSSSDSKISYETFKKHILNLA | |
| | KGKGRISKTKKEYLLEERDINRFSVQKDFINRNLVDTRYATAGLMNL | |
| | LRSYFRVNNLDVKVKSINGGFTSFLRRKWKFKKERNKGYKHHAEDAL | |
| | IIANADFIFKEWKKLDKAKKVMENQMFEEKQAESMPEIETEQEYKEI | |
| | FITPHQIKHIKDFKDYKYSHRVDKKPNRKLINDTLYSTRKDDKGNTL | |
| | IVNNLNGLYDKDNDKLKKLINKSPEKLLMYHHDPQTYQKLKLIMEQY | |
| | GDEKNPLYKYYEETGNYLTKYSKKDNGPVIKKIKYYGNKLNAHLDIT | |
| | DDYPNSRNKVVKLSLKPYRFDVYLDNGVYKFVTVKNLDVIKKENYYE | |
| | VNSKCYEEAKKLKKISNQAEFIASFYKNDLIKINGELYRVIGVNNDL | |
| | LNRIEVNMIDITYREYLENMNDKRPPHIIKTIASKTQSIKKYSTDIL | |
| | GNLYEVKSKKHPQIIKKG | |

### V. References

The following references are incorporated by reference herein in their entireties:
Hoffman EP, Brown RH, Jr., Kunkel LM. Dystrophin: the protein product of the Duchenne muscular dystrophy locus. Cell. 1987;51(6):919-928.
Koenig M, Hoffman EP, Bertelson CJ, Monaco AP, Feener C, Kunkel LM. Complete cloning of the Duchenne muscular dystrophy (DMD) cDNA and preliminary genomic organization of the DMD gene in normal and affected individuals. Cell. 1987;50(3):509-517.
Gao QQ, McNally EM. The Dystrophin Complex: Structure, Function, and Implications for Therapy. Compr Physiol. 2015;5(3):1223-1239.
Guiraud S, Aartsma-Rus A, Vieira NM, Davies KE, van Ommen GJ, Kunkel LM. The Pathogenesis and Therapy of Muscular Dystrophies. Annu Rev Genomics Hum Genet. 2015;16:281-308.
Cong L, Ran FA, Cox D, et al. Multiplex genome engineering using CRISPR/Cas systems. Science. 2013;339(6121):819-823.
Jinek M, Chylinski K, Fonfara I, Hauer M, Doudna JA, Charpentier E. A programmable dual-RNA-guided DNA endonuclease in adaptive bacterial immunity. Science. 2012;337(6096):816-821.
Mali P, Yang L, Esvelt KM, et al. RNA-guided human genome engineering via Cas9. Science. 2013;339(6121):823-826.
Iyer S, Suresh S, Guo D, et al. Precise therapeutic gene correction by a simple nuclease-induced double-stranded break. Nature. 2019;568(7753):561-565.
Gallagher DN, Haber JE. Repair of a Site-Specific DNA Cleavage: Old-School Lessons for Cas9-Mediated Gene Editing. ACS Chem Biol. 2018;13(2):397-405.
Amoasii L, Hildyard JCW, Li H, et al. Gene editing restores dystrophin expression in a canine model of Duchenne muscular dystrophy. Science. 2018;362(6410):86- 91.
Amoasii L, Long C, Li H, et al. Single-cut genome editing restores dystrophin expression in a new mouse model of muscular dystrophy. Sci Transl Med. 2017;9(418).
Bengtsson NE, Hall JK, Odom GL, et al. Muscle-specific CRISPR/Cas9 dystrophin gene editing ameliorates pathophysiology in a mouse model for Duchenne muscular dystrophy. Nat Commun. 2017;8:14454.
Hakim CH, Wasala NB, Nelson CE, et al. AAV CRISPR editing rescues cardiac and muscle function for 18 months in dystrophic mice. JCI Insight. 2018;3(23).
Long C, Amoasii L, Mireault AA, et al. Postnatal genome editing partially restores dystrophin expression in a mouse model of muscular dystrophy. Science. 2016;351(6271):400-403.
Min YL, Chemello F, Li H, et al. Correction of Three Prominent Mutations in Mouse and Human Models of Duchenne Muscular Dystrophy by Single-Cut Genome Editing. Mol Ther. 2020;28(9):2044-2055.
Min YL, Li H, Rodriguez-Caycedo C, et al. CRISPR-Cas9 corrects Duchenne muscular dystrophy exon 44 deletion mutations in mice and human cells. Sci Adv. 2019;5(3): eaav4324.
Nelson CE, Hakim CH, Ousterout DG, et al. In vivo genome editing improves muscle function in a mouse model of Duchenne muscular dystrophy. Science. 2016;351(6271):403-407.
Nelson CE, Wu Y, Gemberling MP, et al. Long-term evaluation of AAV-CRISPR genome editing for Duchenne muscular dystrophy. Nat Med. 2019;25(3):427-432.
Tabebordbar M, Zhu K, Cheng JKW, et al. In vivo gene editing in dystrophic mouse muscle and muscle stem cells. Science. 2016;351(6271): 407-411.
Zhang Y, Li H, Min YL, et al. Enhanced CRISPR-Cas9 correction of Duchenne muscular dystrophy in mice by a self-complementary AAV delivery system. Sci Adv. 2020;6(8):eaay6812.
Amoasii L, Li H, Zhang Y, et al. In vivo non-invasive monitoring of dystrophin correction in a new Duchenne muscular dystrophy reporter mouse. Nat Commun. 2019;10(1):4537.
Kwon JB, Ettyreddy AR, Vankara A, et al. In vivo Gene Editing of Muscle Stem Cells with Adeno-Associated Viral Vectors in a Mouse Model of Duchenne Muscular Dystrophy. Mol Ther Methods Clin Dev. 2020;19:320-329.
Kleinstiver BP, Prew MS, Tsai SQ, et al. Broadening the targeting range of Staphylococcus aureus CRISPR-Cas9 by modifying PAM recognition. Nat Biotechnol. 2015;33(12):1293-1298.
Aartsma-Rus A, Fokkema I, Verschuuren J, et al. Theoretic applicability of antisense-mediated exon skipping for Duchenne muscular dystrophy mutations. Hum Mutat. 2009;30(3):293-299.
Himeda CL, Chen X, Hauschka SD. Design and testing of regulatory cassettes for optimal activity in skeletal and cardiac muscles. Methods Mol Biol. 2011;709:3-19.
Chemello F, Bassel-Duby R, Olson EN. Correction of muscular dystrophies by CRISPR gene editing. J Clin Invest. 2020;130(6):2766-2776.
Min YL, Bassel-Duby R, Olson EN. CRISPR Correction of Duchenne Muscular Dystrophy. Annu Rev Med. 2019;70:239-255.
Zhang Y, Long C, Bassel-Duby R, Olson EN. Myoediting: Toward Prevention of Muscular Dystrophy by Therapeutic Genome Editing. Physiol Rev. 2018;98(3):1205-1240.
Zuo Z, Liu J. Cas9-catalyzed DNA Cleavage Generates Staggered Ends: Evidence from Molecular Dynamics Simulations. Sci Rep. 2016;5:37584.
Lemos BR, Kaplan AC, Bae JE, et al. CRISPR/Cas9 cleavages in budding yeast reveal templated insertions and strand-specific insertion/deletion profiles. Proc Natl Acad Sci U S A. 2018;115(9):E2040-E2047.
Shen MW, Arbab M, Hsu JY, et al. Predictable and precise template-free CRISPR editing of pathogenic variants. Nature. 2018;563(7733):646-651.
Yin H, Price F, Rudnicki MA. Satellite cells and the muscle stem cell niche. Physiol Rev. 2013;93(1):23-67.
Nance ME, Shi R, Hakim CH, et al. AAV9 Edits Muscle Stem Cells in Normal and Dystrophic Adult Mice. Mol Ther. 2019;27(9):1568-1585.
Duan D. Systemic AAV Micro-dystrophin Gene Therapy for Duchenne Muscular Dystrophy. Mol Ther. 2018;26(10):2337-2356.
Truong DJ, Kuhner K, Kuhn R, et al. Development of an intein-mediated split-Cas9 system for gene therapy. Nucleic Acids Res. 2015;43(13):6450-6458.
Chew WL, Tabebordbar M, Cheng JK, et al. A multifunctional AAV-CRISPR-Cas9 and its host response. Nat Methods. 2016;13(10):868-874.
Hoffman EP, Kunkel LM, Angelini C, Clarke A, Johnson M, Harris JB. Improved diagnosis of Becker muscular dystrophy by dystrophin testing. Neurology. 1989;39(8):1011-1017.
Chemello F, Chai AC, Li H, et al. Precise correction of Duchenne muscular dystrophy exon deletion mutations by base and prime editing. Sci Adv. 2021;7(18).
Ran FA, Cong L, Yan WX, et al. In vivo genome editing using Staphylococcus aureus Cas9. Nature. 2015;520(7546):186-191.
Zhang Y, Long C, Li H, et al. CRISPR-Cpf1 correction of muscular dystrophy mutations in human cardiomyocytes and mice. Sci Adv. 2017;3(4):e1602814.
Atmanli A, Hu D, Deiman FE, et al. Multiplex live single-cell transcriptional analysis demarcates cellular functional heterogeneity. Elife. 2019;8.
Brinkman EK, Chen T, Amendola M, van Steensel B. Easy quantitative assessment of genome editing by sequence trace decomposition. Nucleic Acids Res. 2014;42(22):e168.

1. A method of gene editing comprising delivering to a cell a composition comprising a nucleic acid encoding an saCas9, an sgRNA or multiple copies of the same sgRNA, and an AAV vector.

2. The method of claim 1, wherein the SaCas9 is a KKH variant.

3. The method of claim 2, wherein the KKH variant of SaCas9 comprises the amino acid sequence of SEQ ID NO: 41.

4. The method of claim 1, wherein the SaCas9 is a HF variant.

5. The method of claim 4, wherein the HF variant of SaCas9 comprises the amino acid sequence of SEQ ID NO: 42.

6. The method of claim 1, wherein the SaCas9 is a KKH-HF variant.

7. The method of claim 6, wherein the KKH-HF variant of SaCas9 comprises the amino acid sequence of SEQ ID NO: 43.

8. The method of claim 1, further comprising SaCas9 or a nucleic acid encoding the same.

9. The method of claim 8, wherein the SaCas9 comprises the amino acid sequence of SEQ ID NO: 40.

10. The method of any one of the preceding claims, wherein the sgRNA is modified.

11. The method of claim 10, wherein the modification alters one or more 2′ positions and/or phosphodiester linkages.

12. The method of claim 10, wherein the modification alters one or more, or all, of the first three nucleotides of the guide RNA.

13. The method of claim 10, wherein the modification alters one or more, or all, of the last three nucleotides of the guide RNA.

14. The method of claim 10, wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.

15. The method of claim 1, wherein the system further comprises a pharmaceutically acceptable excipient.

16. The method of claim 1, wherein the system is associated with a viral vector.

17. The method of claim 1, wherein the system is associated with a viral vector, wherein the viral vector is an adeno-associated virus (AAV) vector, and wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

18. The method of claim 1, wherein the system is associated with a viral vector, wherein the viral vector is an adeno-associated virus (AAV) vector, and wherein the AAV vector is an AAV serotype 9 (AAV9) vector.

19. The method of claim 1, wherein the system is associated with a viral vector, wherein the viral vector is an adeno-associated virus (AAV) vector, and wherein the AAV vector is an AAVrh10 vector.

20. The method of claim 1, wherein the system is associated with a viral vector, wherein the viral vector is an adeno-associated virus (AAV) vector, and wherein the AAV vector is an AAVrh74 vector.

21. The method of claim 1, wherein the system is associated with a viral vector, wherein the viral vector comprises a tissue-specific promoter.

22. The method of claim 1, wherein the system is associated with a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.

23. The method of claim 1, wherein the system is associated with a viral vector, wherein the viral vector comprises any one or more of the following promoters: U6, H1, and 7SK promoter.

24. The method of claim 1, further comprising a scaffold sequence.

25. The method of claim 24, wherein the scaffold sequence for the sgRNA comprises the sequence of SEQ ID NO: 39.

26. A composition comprising a single-molecule guide RNA (sgRNA) comprising a spacer sequence, or a nucleic acid encoding the sgRNA, wherein:
a) the spacer sequence comprises the reverse complement of the “sgRNA DMD Ex51” shown in FIG. 1B; or
b) the spacer sequence recognizes a 5′-AACAGT-3′ PAM in exon 51 as shown in FIG. 1B; or
c) the spacer sequence comprises ACTCTGGTGACACAACCTGTG (SEQ ID NO: 37); or
d) the sgRNA targets TGAGACCACTGTGTTGGACAC (SEQ ID NO: 38); or
e) the sgRNA generates a DNA double-stand break 4-bp upstream of the premature termination codon as shown in FIG. 1B.

27. The composition of claim 26, further comprising a KKH variant of SaCas9 or a nucleic acid encoding the same.

28. The composition of claim 27, wherein the KKH variant of SaCas9 comprises the amino acid sequence of SEQ ID NO: 41.

29. The composition of claim 26, further comprising a HF variant of SaCas9 or a nucleic acid encoding the same.

30. The composition of claim 29, wherein the HF variant of SaCas9 comprises the amino acid sequence of SEQ ID NO: 42.

31. The composition of claim 26, further comprising a KKH-HF variant of SaCas9 or a nucleic acid encoding the same.

32. The composition of claim 31, wherein the KKH-HF variant of SaCas9 comprises the amino acid sequence of SEQ ID NO: 43.

33. The composition of claim 26, further comprising SaCas9 or a nucleic acid encoding the same.

34. The composition of claim 33, wherein the SaCas9 comprises the amino acid sequence of SEQ ID NO: 40.

35. The composition of claim 26, wherein the sgRNA is modified.

36. The composition of claim 35, wherein the modification alters one or more 2′ positions and/or phosphodiester linkages.

37. The composition claim 35, wherein the modification alters one or more, or all, of the first three nucleotides of the guide RNA.

38. The composition of claim 35, wherein the modification alters one or more, or all, of the last three nucleotides of the guide RNA.

39. The composition of claim 35, wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.

40. The composition of claim 26, wherein the composition further comprises a pharmaceutically acceptable excipient.

41. The composition of claim 26, wherein the composition is associated with a viral vector.

42. The composition of claim 26, wherein the composition is associated with a viral vector, wherein the viral vector is an adeno-associated virus (AAV) vector, and wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

43. The composition of claim 26, wherein the composition is associated with a viral vector, wherein the viral vector is an adeno-associated virus (AAV) vector, and wherein the AAV vector is an AAV serotype 9 (AAV9) vector.

44. The composition of claim 26, wherein the composition is associated with a viral vector, wherein the viral vector is an adeno-associated virus (AAV) vector, and wherein the AAV vector is an AAVrh10 vector.

45. The composition of claim 26, wherein the composition is associated with a viral vector, wherein the viral vector is an adeno-associated virus (AAV) vector, and wherein the AAV vector is an AAVrh74 vector.

46. The composition of claim 26, wherein the composition is associated with a viral vector, wherein the viral vector comprises a tissue-specific promoter.

47. The composition of claim 26, wherein the composition is associated with a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.

48. The composition of claim 26, wherein the composition is associated with a viral vector, wherein the viral vector comprises any one or more of the following promoters: U6, H1, and 7SK promoter.

49. The composition of claim 26, further comprising a scaffold sequence.

50. The composition of claim 49, wherein the scaffold sequence for the sgRNA comprises the sequence of SEQ ID NO: 39.

51. A method of treating Duchenne Muscular Dystrophy (DMD), the method comprising delivering to a cell the composition of any one of the preceding claims.

52. A method of treating Duchenne Muscular Dystrophy (DMD), the method comprising delivering to a cell a composition comprising a single-molecule guide RNA (sgRNA) comprising a spacer sequence, or a nucleic acid encoding the sgRNA, wherein:
a) the spacer sequence comprises the reverse complement of the “sgRNA DMD
b) Ex51” shown in FIG. 1B; or
c) the spacer sequence recognizes a 5′-AACAGT-3′ PAM in exon 51 as shown in FIG. 1B; or
d) the spacer sequence comprises ACTCTGGTGACACAACCTGTG (SEQ ID NO: 37); or
e) the sgRNA targets TGAGACCACTGTGTTGGACAC (SEQ ID NO; 38); or
f) the sgRNA generates a DNA double-stand break 4-bp upstream of the premature termination codon as shown in FIG. 1B.

53. The method of claim 51, wherein the composition is delivered to the cell on a single vector.

54. The method of claim 51, further comprising a KKH variant of SaCas9 or a nucleic acid encoding the same.

55. The method of claim 54, wherein the KKH variant of SaCas9 comprises the amino acid sequence of SEQ ID NO: 41.

56. The method of claim 51, further comprising a HF variant of SaCas9 or a nucleic acid encoding the same.

57. The method of claim 56, wherein the HF variant of SaCas9 comprises the amino acid sequence of SEQ ID NO: 42.

58. The method of claim 51, further comprising a KKH-HF variant of SaCas9 or a nucleic acid encoding the same.

59. The method of claim 58 wherein the KKH-HF variant of SaCas9 comprises the amino acid sequence of SEQ ID NO: 43.

60. The method of claim 51, further comprising SaCas9 or a nucleic acid encoding the same.

61. The method of claim 60, wherein the SaCas9 comprises the amino acid sequence of SEQ ID NO: 40.


SEQUENCE LISTING (44 sequences)

SEQ ID NO: 1 - DNA, 21 bp, Artificial sequence
  Synthetic primer
ccccgtcgtgaagagaagctt

SEQ ID NO: 2 - DNA, 24 bp, Artificial sequence
  Synthetic primer
tctattaggcttcttgtccacccg

SEQ ID NO: 3 - DNA, 50 bp, Artificial sequence
  Synthetic primer
agaagaaatataagactcgagttaattaatgcccatgtaaggaggcaagg

SEQ ID NO: 4 - DNA, 35 bp, Artificial sequence
  Synthetic primer
gcttcttctttggggccatggtggcgctagcgctg

SEQ ID NO: 5 - DNA, 21 bp, Artificial sequence
  Synthetic primer
atttccctggcaaggtctgaa

SEQ ID NO: 6 - DNA, 20 bp, Artificial sequence
  Synthetic primer
atcctcaaggtcacccacca

SEQ ID NO: 7 - DNA, 26 bp, Artificial sequence
  Synthetic primer
caccgcacaggttgtgtcaccagagt

SEQ ID NO: 8 - DNA, 26 bp, Artificial sequence
  Synthetic primer
agacactctggtgacacaacctgtgc

SEQ ID NO: 9 - DNA, 25 bp, Artificial sequence
  Synthetic primer
cccagaagagcaagataaacttgaa

SEQ ID NO: 10 - DNA, 23 bp, Artificial sequence
  Synthetic primer
ctctgttccaaatcctgcattgt

SEQ ID NO: 11 - DNA, 25 bp, Artificial sequence
  Synthetic primer
cctgaaaattccccaatacttgtgc

SEQ ID NO: 12 - DNA, 25 bp, Artificial sequence
  Synthetic primer
gtgtttcaccctaacttttcatccc

SEQ ID NO: 13 - DNA, 26 bp, Artificial sequence
  Synthetic primer
caccgcacagattgtgtcactagagt

SEQ ID NO: 14 - DNA, 26 bp, Artificial sequence
  Synthetic primer
agacactctagtgacacaatctgtgc

SEQ ID NO: 15 - DNA, 58 bp, Artificial sequence
  Synthetic primer
tcgtcggcagcgtcagatgtgtataagagacagcctgaaaattccccaatacttgtgc

SEQ ID NO: 16 - DNA, 55 bp, Artificial sequence
  Synthetic primer
gtctcgtgggctcggagatgtgtataagagacagtccaagctcggttgaagtctg

SEQ ID NO: 17 - DNA, 60 bp, Artificial sequence
  Synthetic primer
tcgtcggcagcgtcagatgtgtataagagacagtcatgaataagagtttggctcaaattg

SEQ ID NO: 18 - DNA, 60 bp, Artificial sequence
  Synthetic primer
gtctcgtgggctcggagatgtgtataagagacaggtgggaaatggtctaggagagtaaag

SEQ ID NO: 19 - DNA, 59 bp, Artificial sequence
  Synthetic primer
tcgtcggcagcgtcagatgtgtataagagacagcatcattccatatctatcacctagcc

SEQ ID NO: 20 - DNA, 58 bp, Artificial sequence
  Synthetic primer
gtctcgtgggctcggagatgtgtataagagacagcttcagagaattgaccaaccactg

SEQ ID NO: 21 - DNA, 60 bp, Artificial sequence
  Synthetic primer
tcgtcggcagcgtcagatgtgtataagagacagccaattctctgtatcaaatcttcttgg

SEQ ID NO: 22 - DNA, 56 bp, Artificial sequence
  Synthetic primer
gtctcgtgggctcggagatgtgtataagagacaggcaggctgggaatatcaggttc

SEQ ID NO: 23 - DNA, 58 bp, Artificial sequence
  Synthetic primer
tcgtcggcagcgtcagatgtgtataagagacaggagtcagaatttcaccatgttagcc

SEQ ID NO: 24 - DNA, 56 bp, Artificial sequence
  Synthetic primer
gtctcgtgggctcggagatgtgtataagagacagttccctttaacccaagaccctg

SEQ ID NO: 25 - DNA, 53 bp, Artificial sequence
  Synthetic primer
tcgtcggcagcgtcagatgtgtataagagacaggtgtgtgggtggggaagaat

SEQ ID NO: 26 - DNA, 54 bp, Artificial sequence
  Synthetic primer
gtctcgtgggctcggagatgtgtataagagacagtcacagaggtagcctggaca

SEQ ID NO: 27 - DNA, 55 bp, Artificial sequence
  Synthetic primer
tcgtcggcagcgtcagatgtgtataagagacagagcacatggttatccttgacgt

SEQ ID NO: 28 - DNA, 59 bp, Artificial sequence
  Synthetic primer
gtctcgtgggctcggagatgtgtataagagacagctttcattccttgaatctgacttca

SEQ ID NO: 29 - DNA, 53 bp, Artificial sequence
  Synthetic primer
tcgtcggcagcgtcagatgtgtataagagacagacggactctcaaactgctcc

SEQ ID NO: 30 - DNA, 55 bp, Artificial sequence
  Synthetic primer
gtctcgtgggctcggagatgtgtataagagacaggccctcattgctgaccttctc

SEQ ID NO: 31 - DNA, 57 bp, Artificial sequence
  Synthetic primer
tcgtcggcagcgtcagatgtgtataagagacagcgaaggattctattgcctttggtg

SEQ ID NO: 32 - DNA, 60 bp, Artificial sequence
  Synthetic primer
gtctcgtgggctcggagatgtgtataagagacagacttctgcttacaccttcacatagtg

SEQ ID NO: 33 - DNA, 55 bp, Artificial sequence
  Synthetic primer
tcgtcggcagcgtcagatgtgtataagagacagcacctgtgcccaccttatagtg

SEQ ID NO: 34 - DNA, 59 bp, Artificial sequence
  Synthetic primer
gtctcgtgggctcggagatgtgtataagagacagctttgattagagacaagggacccag

SEQ ID NO: 35 - DNA, 43 bp, Artificial sequence
  Synthetic primer
aatgatacggcgaccaccgagatctacactcgtcggcagcgtc

SEQ ID NO: 36 - DNA, 45 bp, Artificial sequence
  Synthetic primer
  misc_feature (25)..(30): n is a, c, g, or t
caagcagaagacggcatacgagatnnnnnngtctcgtgggctcgg

SEQ ID NO: 37 - DNA, 21 bp, Artificial sequence
  Synthetic nucleic acid
actctggtgacacaacctgtg

SEQ ID NO: 38 - DNA, 21 bp, Artificial sequence
  Synthetic nucleic acid
tgagaccactgtgttggacac

SEQ ID NO: 39 - DNA, 87 bp, Artificial sequence
  Synthetic nucleic acid
gtttaagtactctgtgctggaaacagcacagaatctacttaaacaaggcaaaatgccgtgtttatctcgtcaacttgttggcgagat

SEQ ID NO: 40 - protein, 1052 aa, Artificial sequence
  Synthetic amino acid
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 41 - protein, 1052 aa, Artificial sequence
  Synthetic amino acid
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 42 - protein, 1052 aa, Artificial sequence
  Synthetic amino acid
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELASVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDAQIAIFARLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 43 - protein, 1052 aa, Artificial sequence
  Synthetic amino acid
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELASVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDAQIAIFARLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 44 - DNA, 3156 bp, Artificial sequence
  Synthetic nucleic acid
aagcgcaattacatcctgggcctggatatcggcatcacctccgtgggctacggcatcatcgactatgagacacgggatgtgatcgacgccggcgtgagactgttcaaggaggccaacgtggagaacaatgagggccggcggagcaagaggggagcaaggcgcctgaagcggagaaggcgccacagaatccagagagtgaagaagctgctgttcgattacaacctgctgaccgaccactccgagctgtctggcatcaatccttatgaggcccgggtgaagggcctgtcccagaagctgtctgaggaggagttttctgccgccctgctgcacctggcaaagaggagaggcgtgcacaacgtgaatgaggtggaggaggacaccggcaacgagctgagcacaaaggagcagatcagccgcaattccaaggccctggaggagaagtatgtggccgagctgcagctggagcggctgaagaaggatggcgaggtgaggggctccatcaatcgcttcaagacctctgactacgtgaaggaggccaagcagctgctgaaggtgcagaaggcctaccaccagctggatcagagctttatcgatacatatatcgacctgctggagaccaggcgcacatactatgagggaccaggagagggctcccccttcggctggaaggacatcaaggagtggtacgagatgctgatgggccactgcacctattttccagaggagctgagatccgtgaagtacgcctataacgccgatctgtacaacgccctgaatgacctgaacaacctggtcatcaccagggatgagaacgagaagctggagtactatgagaagttccagatcatcgagaacgtgttcaagcagaagaagaagcctacactgaagcagatcgccaaggagatcctggtgaacgaggaggacatcaagggctaccgcgtgaccagcacaggcaagccagagttcaccaatctgaaggtgtatcacgatatcaaggacatcacagcccggaaggagatcatcgagaacgccgagctgctggatcagatcgccaagatcctgaccatctatcagagctccgaggacatccaggaggagctgaccaacctgaatagcgagctgacacaggaggagatcgagcagatcagcaatctgaagggctacaccggcacacacaacctgtccctgaaggccatcaatctgatcctggatgagctgtggcacacaaacgacaatcagatcgccatctttaacaggctgaagctggtgccaaagaaggtggacctgagccagcagaaggagatcccaaccacactggtggacgatttcatcctgtcccccgtggtgaagcggagcttcatccagagcatcaaagtgatcaacgccatcatcaagaagtacggcctgcccaatgatatcatcatcgagctggccagggagaagaactctaaggacgcccagaagatgatcaatgagatgcagaagaggaaccgccagaccaatgagcggatcgaggagatcatcagaaccacaggcaaggagaacgccaagtacctgatcgagaagatcaagctgcacgatatgcaggagggcaagtgtctgtatagcctggaggccatccctctggaggacctgctgaacaatccattcaactacgaggtggatcacatcatcccccggagcgtgagcttcgacaattcctttaacaataaggtgctggtgaagcaggaggagaactctaagaagggcaataggacccctttccagtacctgtctagctccgattctaagatcagctacgagaccttcaagaagcacatcctgaatctggccaagggcaagggccgcatctctaagaccaagaaggagtacctgctggaggagcgggacatcaacagattcagcgtgcagaaggacttcatcaaccggaatctggtggacaccagatacgccacacgcggcctgatgaatctgctgcggtcctatttcagagtgaacaatctggatgtgaaggtgaagagcatcaacggcggcttcacctcctttctgcggagaaagtggaagtttaagaaggagagaaacaagggctataagcaccacgccgaggatgccctgatcatcgccaatgccgacttcatctttaaggagtggaagaagctggacaaggccaagaaagtgatggagaaccagatgttcgaggagaagcaggccgagagcatgcccgagatcgagaccgagcaggagtacaaggagattttcatcacacctcaccagatcaagcacatcaaggacttcaaggactacaagtattcccacagggtggataagaagcccaaccgcgagctgatcaatgacaccctgtattctacaaggaaggacgataagggcaataccctgatcgtgaacaatctgaacggcctgtacgacaaggataatgacaagctgaagaagctgatcaacaagagccccgagaagctgctgatgtaccaccacgatcctcagacatatcagaagctgaagctgatcatggagcagtacggcgacgagaagaacccactgtataagtactatgaggagaccggcaactacctgacaaagtattccaagaaggataatggccccgtgatcaagaagatcaagtactatggcaacaagctgaatgcccacctggacatcaccgacgattaccccaacagccggaataaggtggtgaagctgagcctgaagccatacaggttcgacgtgtacctggacaacggcgtgtataagtttgtgacagtgaagaatctggatgtgatcaagaaggagaactactatgaagtgaatagcaagtgctacgaggaggccaagaagctgaagaagatcagcaaccaggccgagttcatcgcctctttttacaacaatgacctgatcaagatcaatggcgagctgtatagagtgatcggcgtgaacaatgatctgctgaaccgcatcgaagtgaatatgatcgacatcacctaccgggagtatctggagaacatgaatgataagaggccccctcgcatcatcaagaccatcgcctctaagacacagagcatcaagaagtactctacagacatcctgggcaacctgtatgaggtgaagagcaagaagcaccctcagatcatcaagaagggc